(19) United States
(12) Patent Application Publication
(10) Pub. No.: US 2014/0283215 A1
(43) Pub. Date: Sep. 18, 2014

(54) METHODS FOR INCREASING STARCH CONTENT IN PLANT COBS

(71) Applicant: Syngenta Participations AG, Basel (CH)

(72) Inventor: John Steffens, Durham, NC (US)

(73) Assignee: SYNGENTA PARTICIPATIONS AG, Basel (CH)

(21) Appl. No.: 14/293,780

(22) Filed: Jun. 2, 2014

Related U.S. Application Data
(63) Continuation of application No. 13/203,231, filed on Nov. 16, 2011.
(60) Provisional application No. 61/155,176, filed on Feb. 25, 2009.

(51) Int. Cl. C12N 15/82

(52) U.S. Cl. CPC C12N 15/8245
USPC 800/284

(57) ABSTRACT

Methods and compositions for increasing the starch content in cob tissues of a plant are provided. The method comprises down-regulating the activity of starch degradation enzymes in a plant. The resulting transgenic plants of the invention have increased starch content in cob tissues. In one embodiment the method involves manipulating a monocot plant to down-regulate the activity of a starch degradation enzyme in cob tissues. The plants are useful for improving the yield of free sugars from plant biomass.

### RELATED APPLICATIONS

This application is a continuation of U.S. application Ser. No. 13/203,231 filed Nov. 16, 2011, which claims priority to PCT/US10/25129 filed Feb. 24, 2010, which claims priority to 61/155,176 filed Feb. 25, 2009, which is herein incorporated by reference in their entireties.

### FIELD OF THE INVENTION

This invention relates to plant molecular biology, particularly to methods and compositions for increasing starch accumulation in plant cobs and the use of these plant tissues in commercial applications.

### BACKGROUND OF THE INVENTION

Plant biomass is comprised of sugars and represents the greatest source of renewable hydrocarbon on earth. Unlike other renewable energy sources, biomass can be converted directly into liquid fuels. The two most common types of biofuels are ethanol (ethyl alcohol) and biodiesel. Ethanol is an alcohol, which can be produced by fermenting any biomass high in carbohydrates (starches, sugars, or celluloses). Once fermentable sugars have been obtained from the biomass material, these sugars can then be fermented to produce ethanol through a process similar to brewing beer. However, this enormous resource is under-utilized due to the fact sugars are locked in complex polymers, which are often referred to collectively as lignocellulose.

Conventional breakdown of the lignocellulose into monomers (monosaccharides) requires the biomass source material to be softened through chemical and/or physical pre-treatments. Enzymes may also be added that hydrolyze the polymeric forms of sugars contained in the biomass into monosaccharides. Subsequent fermentation can then be carried out utilizing both the 6-carbon and 5-carbon sugars to produce ethanol or other desired bio-products. Sugars generated from the degradation of plant biomass could provide plentiful, economically competitive feedstocks for fermentation into chemicals, plastics, feed additives and fuels.

Carbohydrates constitute the most abundant organic compounds on earth. They are principally found in plants as complex glucose polymers either in the form of cellulose or starch. Cellulose, hemicellulose and glucans make up many structural components of the plant cell wall and woody tissues. These structural components are often complexed with other molecules such as proteins, fats and lignin. Starch is utilized by the plant as a principle storage carbohydrate in seeds and grain consisting of essentially pure linked glucose polymers. Starches are found in many grains as well as in tubers and roots. Starch is a desirable storage carbohydrate due to the fact that it is compositionally simple and can be readily broken down by the plant for energy. Comparatively, lignocellulosic material is composed of glucose and/or several different sugars complexed with lignin. Starch is readily hydrolysable to monomer sugars via effective and inexpensive starch-hydrolysing enzymes whereas lignocellulosic material is neither readily hydrolysable nor relatively inexpensive to process. Carbohydrates are also found in abundance in the form of the simple disaccharide sucrose. Sucrose may be found in crops such as sugarcane, sugarbeets, and sweet sorghum. Unlike sucrose, starch is stable and can be stored in dehydrated form for long periods of time.

Plant cob primarily consists of lignocellulose and is generally discarded as waste in most agricultural practices. For example, combines which harverst corn, first strip cob of their kernels and then discharge the stripped cob back into the field as waste. Maize cob makes up approximately 15-18% of total above ground maize plant biomass. As the current price of corn has increased, supplies of corn have been very limited and there has been a drive to produce fuels from less expensive non-food sources or waste products. Currently the U.S. produces approximately 8.6 billion gallons of ethanol per year with the vast majority of the ethanol being derived from corn starch. It has been predicted that the cellulosic fermentation of cob could add an additional 5 billion gallons of ethanol to the U.S's total ethanol output. Other estimates predict that use of corn cob in fermentations may increase total corn ethanol yield by 11% per bushel or 27% per acre. However, the fermentation of cob to fuel is limited by cost and less than efficient methods. Current cob fermentation practices require that the cob be pretreated with a mixture of enzymes that assists in the breakdown of cob's lignocellulose and fiber structure. Thermochemical conditions may also be employed as well to assist in the breakdown of cob (e.g., high heat, basic pH, etc.). Following pretreatment, sugars are then extracted from the pretreated cob and in some instances additional enzymes may be added to further degrade the sugars into smaller sugars. These sugars are then converted to ethanol via yeast fermentation. Current cob fermentation costs have been estimated to be approximately $1 more per gallon to produce compared to commercial corn starch ethanol fementations. The structural lignocellulosic composition of cob poses several limitations on current cob fermentation practices. One limitation of current cob fermentation practices may be the need for pretreatment processes used to extract sugars from cob may be both costly and timely. Another limitation of current cob fermentation practices may be the need to extract the appropriate sugars or perform enzyme treatments to further degrade complex sugars into simple sugars that can be readily converted by yeast to ethanol. It may also be necessary to find or create optimal yeast strains able to utilize specific sugar profiles derived from pretreatments and enzyme hydrolysis. Another limitation may be that methods of extracting sugar from cob are inefficient due to the dense structure of lignocellulose.

It would be desirable to produce cob tissues which are beneficial in the production of monomer sugars where a higher proportion of carbohydrate is in the form of starch. Methods for creating starch-rich cob biomass and methods for generating free sugars and oligosaccharides from cob biomass as well as use of these free sugars in the production of chemicals, plastics, feed additives and fuels are provided.

### SUMMARY OF THE INVENTION

Compositions and methods for increasing the starch content in cob tissues of plants are provided. Further provided, are methods in which cobs containing increased amounts of starch may be used in biomass conversion methods as well as in animal feed applications. The method involves independently or jointly down-regulating the endogenous activity of enzymes involved in the plant transitory starch degradation pathway. Down-regulation may be targeted constitutively throughout the plant or within preferred target tissues (i.e. stem, leaf, cob, etc.). The transgenic plants of the invention have increased starch content in cob tissues. The methods described herein may be beneficial in increasing the value of plant cob tissue in the use of producing biofuels and animal feed applications. The cobs obtained from these transgenic plants can be converted to generate an enhanced level of free sugars that are useful in the downstream fermentation of free sugars into chemicals, plastics, feed additives and fuels. Also provided are methods of producing a self-processing cob with increased starch content wherein the plant or plant part expresses a processing enzyme (e.g., alpha-amylase, glucoamylase, cellulases, CBHI, etc.) wherein the processing enzyme is targeted away from its relative substrate and that upon activation (e.g. milling, addition of water, pH, temperature adjustment) of the processing enzyme(s) (mesophilic, thermophilic, or hyperthermophilic) the plant or plant part is capable of self-processing the substrate upon which it acts in order to obtain the desired result.

### DETAILED DESCRIPTION OF THE INVENTION

### Overview

The article “a” and “an” are used herein to refer to one or more than one (i.e., to at least one) of the grammatical object of the article. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Methods and compositions for increasing the starch content in cob tissues of a plant are provided. The method comprises down-regulating the activity of enzymes involved in the transitory starch degradation pathway in a plant. The resulting transgenic plants of the current invention have increased starch content in cob tissues. Further provided, are methods of use for plants with increased starch content in cob tissues.

Transgenic plants, seeds, plant tissues and plant parts are provided. It is recognized that the process may be controlled by the use of constitutive, tissue, temporal or chemically regulated promoters. The following embodiments can be carried out in cobs of monocotyledon plants and analogous structures found in either monocotyledon or dicotyledon plants.

A method to increase starch in cob tissue may be desirable across multiple industries for example but not limited to ethanol, animal feed, plastics, chemicals and other industrial applications. One embodiment of current application involves manipulating a plant to down-regulate the activity of one or more chloroplastic or cytosolic enzymes involved in the transitory starch degradation pathway, herein referred to as “starch degradation enzymes”. The resultant plants of the invention have increased starch content in cob tissues. Starch degradation enzymes include, but are not limited to alpha-amylase, glucan water dikinase, phosphoglucan water dikinase, limit dextrinase, isoamylase, beta-amylase, glucan phosphorylase, disproportionating enzyme, chloroplastic maltose transporter protein, chloroplastic glucose transporter protein, chloroplastic triose phosphate transporter protein, cytosolic transglucosidase, glucan phosphorylase, phosphoglucan phosphatase (starch excess 4) and hexokinase.

The methods of the invention find use in the integration of current practices for the cultivation of crop plants for the purpose of obtaining a commercially desired plant material with increased starch accumulation in the cob tissues of the crop plants, and the use of the crop plant cobs as a source of biomass for the production of fermentable sugars, or for agricultural and/or human consumption. The modified plants and plant parts can be used in the production of alcohol and yield increased ethanol by engineering the plant cob to accumulate starch.

As used herein, “crop plant” refers to any plant that is cultivated for the purpose of producing plant material sought after by man or animal for either oral consumption, or for utilization in an industrial, pharmaceutical, or commercial process. The invention may be applied to any variety of plants, including, but not limited to maize, wheat, rice, barley, soybean, cotton, sorghum, beans in general, rape/canola, alfalfa, flax, sunflower, safflower, millet, rye, sugarcane, sugar beet, cocoa, tea, tropical sugar beet, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indiangrass, Big bluestem grass, miscanthus and the like. It is recognized that mixtures of plants may be used.

As used herein, the term “energy crop” refers to crops that may be favorable to use in a biomass conversion method in converting plant biomass to fuels. This group comprises but is not limited to sugarcane, sugarbeet, sorghum, switchgrass, miscanthus, wheat, rice, oat, barley and maize.

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

In one embodiment, the plant is an indeterminate plant. These varieties grow vegetatively for indefinite periods in temperate regions. An indeterminate plant can be engineered to accumulate starch in cob tissues and can be grown until the first frost. At that time, the plant could be allowed to desiccate, harvested dry, and used for food, livestock feed, or in a biomass conversion processes.

As used herein, “biomass” refers to useful biological material including a product of interest, which material is to be collected and is intended for further processing to isolate or concentrate the product of interest. “Biomass” may comprise the fruit or parts of it or seeds, leaves, or stems, cob or roots where these are the parts of the plant that are of particular interest for the industrial purpose. “Biomass”, as it refers to plant material, includes any structure or structures of a plant that contain or represent the product of interest.

An increase in cob starch accumulation may be desirable, for example, in cob tissue which is conserved by silage or drying. In one embodiment it may be beneficial to ensilage a plant with increased cob starch. In another embodiment, it may be beneficial to engineer a plant with both increased cob starch and increased green tissue starch such as is described in U.S. Publication US 2009/0119800-A1 herein incorporated by reference and use said plant in silage. In another embodiment it may be beneficial to produce said plant with increased cob starch wherein the plant has been engineered to express or has been treated with a phytase. In another embodiment it may be beneficial to produce said plant engineered to express phytase or treated with phytase to additionally be engineered to express an amylase. As used herein, the term “amylase” encompasses enzymes (e.g., E.C. class 3.2.1.1) having α-amylase activity, for example, α-amylases capable of hydrolyzing internal α-1,4-glucan links in polysaccharides, including amylase enzymes capable of hydrolyzing starch to sugars at alkaline pHs or at acidic pHs. These enzymes have also been described as those effecting the exohydrolysis or endohydrolysis of 1,4-α-D-glucosidic linkages in polysaccharides containing 1,4-α-linked D-glucose units. Another term used to describe these enzymes is “glycogenase.” US Patent Publication 2003/0125534 and U.S. Patent Publication 2004/0018607 (both are herein incorporated by reference) describe numerous α-amylase enzymes that may be used in various embodiments of the invention. Methods for making and using organisms expressing α-amylase enzymes (for example, to produce fermentable substrates for the production of ethanol) are also provided in U.S. Patent Publication No. 2003/0135885, which is herein incorporated by reference in its entirety. The term “harvest index” as defined herein refers to the ratio of biomass yield to the cumulative biomass at harvest. Two of the best energy crops today, cane and beets, in terms of harvest index, have limitations on storage stability, and have high moisture content at harvest. High moisture content has several disadvantages such as transportation costs for the harvest are higher since a greater proportion of the water needs to be moved with the crop. Storage stability is a significant issue, since there may be continued metabolism, or microbial contaminations that can lead to crop spoilage and sugar loss. Perishability of the crop has very different infrastructural implications for the movement, storage, and utilization of these types of agricultural products. An increase of the starch content would lead to a considerable increase of dry substance and storage stability.

One embodiment of the current application provides a method for increasing levels of starch in plant cob tissue comprising inserting an expression cassette into a plant cell comprising a polynucleotide wherein expression of the polynucleotide sequence decreases or inhibits the activity of one or more starch degradation enzymes selected from the group consisting of alpha-amylase, glucan water dikinase, phosphoglucan water dikinase, limit dextrinase, isoamylase, beta-amylase, chloroplastic glucan phosphorylase, disproportionating enzyme, chloroplastic maltose transporter protein (Mex1), phosphoglucan phosphatase (starch excess 4), chloroplastic glucose transporter protein, and chloroplastic triose phosphate transporter protein. Regeneration of transgenic plants from the plant cell comprising an expression cassette wherein expression of the polynucleotide sequence decreases or inhibits the activity of one or more starch degradation enzymes. The said polynucleotide would preferably be linked to an operable cob tissue preferred promoter such as but not limited to the Oryza sativa MADS-box (OsMADS) promoter such as described in publication US 2007/0006344 herein incorporated by reference. The resultant cob will have an increased level of starch thus having a higher harvest index and commercial value. In a preferred embodiment the starch degradation enzyme is operably linked to a OsMADS13 promoter (GenBank Accession Number AF151693).

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

In some embodiments, the presently disclosed subject matter relates to an expression cassette comprising a 5′-regulatory sequence and a nucleic acid molecule operably linked to said 5′-regulatory sequence, wherein said nucleic acid molecule is heterologous to the 5′-regulatory sequence, and wherein the product of the expression of said nucleic acid molecule is targeted to the cob tissue of a plant. The 5′-regulatory sequence comprises the following regions: a promoter, a first exon, a first intron, and a 5′ portion of a second exon, wherein said 5′-regulatory sequence has been engineered to include a translational initiation codon at the 3′ end of said 5′-regulatory sequence, and not to contain additional translation initiation codons upstream of said translation initiation codon. The presently disclosed subject matter further relates to an expression cassette wherein the 5′ portion of the second exon comprises the first 15 nucleotides from the 5′ end of the exon and a Kozak sequence.

In some embodiments, the presently disclosed subject matter provides an expression cassette comprising SEQ ID NO: 8 operably linked to a heterologous gene. In one aspect the expression cassette comprises a 5′-regulatory sequence and a nucleic acid molecule operably linked to the 5′-regulatory sequence, wherein the nucleic acid molecule is heterologous to the 5′-regulatory sequence. The 5′-regulatory sequence can comprise the following regions: a promoter, a first exon, a first intron, and a 5′ portion of a second exon. The 5′-regulatory sequence is engineered to include a translational initiation codon at its 3′ end, and not to contain additional translation initiation codons upstream of the translation initiation codon. The term “portion” as used here can refer to a sequence from an intron or exon, such as from the 5′ end of exon 2, of a desired length as can be determined by the guidance provided herein including the Examples herein below. By way of example and not limitation, the 5′ portion of the second exon included in the cassette can include the first 15 nucleotides from the 5′ end of the exon. The product of the expression of the nucleic acid molecule can be targeted to the cob tissue of a plant. The expression cassette design is first disclosed in U.S. Application No. 2007/0006344, which is incorporated herein by reference.

### Starch Biosynthesis and Degradation

Starch is one of the most abundant polymers produced in nature and is synthesized as a storage carbohydrate throughout the plant kingdom. In storage organs it serves as a long-term carbon reserve, whereas in photosynthetically competent tissues it is transiently accumulated to provide both reduced carbon and energy during periods unfavorable for photosynthesis. Starch is a desirable storage carbohydrate because it is compositionally simple compared to cellulosic material. Cellulosic material comprises several different sugars, complexed with lignin. Lignocellulose is extremely difficult to break down enzymatically. In contrast, starch is comprised of glucose and is readily hydrolysable to monomer sugars via effective and inexpensive starch-hydrolyzing enzymes. The accumulation of starch in plant cob tissues would provide a rich source for simple sugars in the plant biomass.

Starch degradation in green tissue involves multiple enzymes and transporters. Transitory starch is essentially converted in the chloroplast stroma to glucose, maltose and triose phosphate through the actions of starch degradation enzymes. These sugars are then transported from the chloroplast stroma to the cytosol via sugar transporters. Once in the cytosol these simple sugars will then be utilized in plant cellular metabolism. Prior research was focused on the suppression or inactivation of key starch degradation enzymes and transporters within the chloroplast stroma in order to accumulate starch within green tissues. Surprisingly, it has been found that the downregulation of starch degradation enzymes in cob result in an increase of starch in cob. The plants also exhibit a starch excess phenotype in their green tissues through potential leaky expression of the OsMADs promoter. By “leaky” it is meant that the promoter may preferably direct a gene to express in one tissue type and non-preferably to a lesser degree direct a gene to express in a second tissue. It is known in the art that many promoters exhibit this type of expression profile wherein gene expression is directed to a specific tissue but may express the gene to a lesser degree in other tissue types. A maize “cob preferred promoter”, is any promoter that directs the expression of a gene preferably to the cob of a maize plant and may or may not subsequently express the same gene in other maize plant tissues to a lesser or equal degree. In one embodiment it may be desirable to use a constitutive promoter wherein the starch degradation enzyme is downregulated constitutively throughout the plant.

Starch comprises both linear (amylose) and branched (amylopectin) glucose polymers. Amylopectin from many, but not all plant sources contains phosphate-monoesters that are linked mainly to the C6 and C3 positions of glycosyl residues. The biochemical mechanism of starch phosphorylation has, however, only recently been elucidated. Transgenic potato plants (Lorberth et at (1998) Nat Biotechnol. 16(5):473-7) and the sex1 mutant of Arabidobsis (Yu et al. (2001) Plant Cell 13(8):1907-18) are deficient in a starch associated protein, which is herein referred to as R1, and they synthesize starch with decreased phosphate content. The purified recombinant R1-protein from potato is able to phosphorylate α-glucans (Ritte et al. (2002) Proc Natl Acad Sci USA 99(10):7166-71). It catalyses a dikinase-type reaction, liberating the γ-phosphate of ATP (resulting in the release of orthophosphate), but using the β-phosphate to phosphorylate glucosyl residues of the polyglucan. Because of this activity, the protein is considered a glucan, water dikinase (GWD) (Ritte et al. (2003) Planta 216(5):798-801).

Inhibition of the R1 gene which codes for an R1 protein from potatoes in transgenic potato plants results in a reduction of the phosphate content of the starch which can be isolated from the potato tubers (Lorberth et al.). Furthermore, Lorberth et al. showed that the R1 protein from Solanum tuberosum is capable of phosphorylating bacterial glycogen if the corresponding R1 cDNA is expressed in E. coli (Lorberth et al., Nature Biotech. 16, (1998), 473-477). Ritte et al. (Plant J. 21, (2000), 387-391) showed that the R1 protein from Solanum tuberosum binds reversibly to starch grains in potato plants, wherein the strength of binding to the starch grain depends on the metabolic status of the plant. In starch grain-bound form, the protein in potato plants mainly occurs in leaves which are kept in the dark. After the leaves are illuminated, however, the protein is mainly present in a soluble form which is not bound to starch grains.

The phosphorylation of starch strongly affects its in vivo degradability. This activity is indicated by the starch excess phenotype observed in leaves of GWD deficient potato or Arabidopsis plants (Lorberth et al., 1998, supra; Yu et al., 2001, supra). A reduction in the expression and/or activity of the R1 protein and its homologs in a plant or plant cell result in this starch excess phenotype, which means that a plant deficient in R1 activity is no longer capable of mobilizing the starch synthesized in its photosynthetic or storage tissues (transitory starch). Therefore, these plants show an accumulation of starch in their green tissues. By “green tissues” is intended all of the green structures in a plant, including leaves, stems, and unripened fruit. Surprisingly it has been discovered that the downregulation of starch degradation enzymes in cob results in starch accumulation in cob tissue.

This starch excess property can be assayed, e.g., as described in U.S. Patent Application Publication No. 2006/0236426, herein incorporated by reference. In particular, source leaves of the plants are kept in darkness for different time intervals and then stained with iodine in order to determine their starch content. Leaves of plants which cannot mobilize the transitory starch in the dark show a blue staining, or the blue staining in these leaves is stronger or staining is apparent after longer time intervals in the dark as compared to staining that may occur in leaves of corresponding wild-type plants. Similar methods may be modified to measure starch accumulation in cob tissues.

Furthermore, the accumulation of transitory starch in the cob tissues can also be tested by enzymatically determining the starch content. This can be done, e.g. as described in Muller-Rober et al. (EMBO J. 11 (1992), 1229-1238). Cob tissues of plants in which the activity of the one or more starch degradation enzymes are reduced preferably have an increased starch content of at least about 50%, at least about 75%, at least about 100%, at least about 150%, at least about 200%, at least about 250%, at least about 300%, at least about 350%, at least about 400%, at least 600%, or greater, when compared to cob tissues of corresponding wild-type plants.

### Inhibition of Starch Degradation Enzyme Activity

In the methods and compositions of the present invention, starch accumulation occurs in the cob tissues of plants in which the activity of the starch degradation enzymes or homologs thereof are down-regulated. By down-regulating the activity, the level of activity of the starch degradation protein or enzyme in a plant is decreased or completely suppressed in comparison to the activity in a corresponding control plant which has not been manipulated to decrease the activity of a starch degradation enzyme. The activity of the starch degradation enzyme, the target protein, is inhibited, reduced, or eliminated if the activity is less than 95%, less than about 90%, less than about 80%, less than about 70%, less than about 60%, less than about 50%, less than about 40%, less than about 30%, less than about 20%, less than about 10%, or is 100% less than the activity of the starch degradation enzyme in a plant that is not a mutant or that has not been genetically modified to inhibit the expression of a starch degradation enzyme. The activity of a starch degradation enzyme can be measured by measuring the starch content in plant cob tissue. Methods for the measurement of starch content are available in the art. See, for example, Yu et al. (2001) The Plant Cell 13:1907-1918. R1 enzyme activity can be measured by methods set forth, for example, in Ritte et al. (2002) Proc. Natl. Acad. Sci USA 14:7166-7171. Likewise, levels of expression of the starch degradation enzymes can be directly measured by immunoblots demonstrating a reduction in a starch degradation enzyme in the plant, by Western blot analysis and the like.

Any method to reduce the activity of a starch degradation enzyme in a plant can be used in the practice of the methods of the invention. For example, the activity and/or level of the R1 protein can be reduced or eliminated by introducing into a plant a polynucleotide that inhibits the level or activity of the R1 protein. The polynucleotide may inhibit the expression or the translation of the messenger RNA. Likewise, down-regulation may be achieved by transforming the plant with a nucleic acid sequence that encodes a polypeptide that inhibits the transcription or translation of starch degradation enzyme, or that inhibits the activity of the starch degradation enzyme.

The terms “inhibit,” “inhibition,” “down-regulation” and “inhibiting” as used herein refers to any decrease in the expression or function of a target gene product, including any relative decrement in expression or function up to and including complete abrogation of expression or function of the target gene product. The term “expression” as used herein in the context of a gene product refers to the biosynthesis of that gene product, including the transcription and/or translation and/or assembly of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants.

Methods for inhibiting or eliminating the expression of a gene in a plant are well known in the art, and any such method may be used in the methods of the present invention. Antisense constructions, complementary to at least a portion of the messenger RNA (mRNA) for the target sequence can be utilized. Antisense nucleotides are constructed to hybridize with the corresponding mRNA. Modifications of the antisense sequences may be made as long as the sequences hybridize to and interfere with expression of the corresponding mRNA. In this manner, antisense constructs having at least about 70%, at least about 80%, at least about 85% or higher sequence identity to the corresponding sense sequences may be used. Furthermore, portions of the antisense nucleotides may be used to disrupt the expression of the target gene. Generally, sequences of at least about 10 nucleotides, at least about 20 nucleotides, at least about 30 nucleotides, at least about 40 nucleotides, at least about 50 nucleotides, at least about 100 nucleotides, at least about 200 nucleotides, at least about 300, at least about 400, at least about 450, at least about 500, at least about 550, or greater may be used. Antisense methods are known in the art, See, for example, Sheehy et al. (1988) Proc. Natl. Acad. Sci. USA 85:8805-8809; and U.S. Pat. Nos. 5,107,065; 5,453,566; and 5,759,829); herein incorporated by reference.

Cosuppression may also be used to suppress the expression of the target gene. In this manner, a heterologous starch degradation enzyme sequence is expressed in a plant of interest in the sense orientation to suppress the expression of the endogenous starch degradation enzyme gene in the plant. Methods for cosuppression are known in the art. See, for example, Taylor (1997) Plant Cell 9:1245; Jorgensen (1990) Trends Biotech. 8(12):340-344; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al (2002) Plant Physiol. 129:1723-1731; Yu et al. (2003) Phytochemistry 63:753-763; Flavell (1994) Proc. Natl. Acad. Sci. USA 91:3490-3496; Finnegan et al. (1994) Bio/Technology 12:883-888; Neuhuber et al. (1994) Mol. Gen. Genet. 244:230-241; and U.S. Pat. Nos. 5,034,323, 5,283,184, and 5,942,657; all of which are herein incorporated by reference.

Cosuppression involves transforming plants with a DNA construct comprising a promoter that drives expression in a plant operably linked to at least a portion of a polynucleotide that corresponds to the transcript of the gene of interest or the target gene. The nucleotide sequence is constructed or chosen to have substantial sequence identity to the sequence of the transcript of the endogenous gene, typically greater than about 60% sequence identity, more typically greater than about 80% sequence identity, more typically greater than about 90% sequence identity, and in some instances greater than about 95% sequence identity.

RNA interference (RNAi) can also be used to down-regulate starch degradation enzyme genes. See, generally, Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507. In RNAi, long double-stranded RNAs (dsRNAs), typically >200 nucleotides, can be used to silence the expression of a target gene in a plant. Upon introduction, the long dsRNAs enter a cellular pathway that is commonly referred to as the RNA interference (RNAi) pathway. First, the dsRNAs are processed into 20-25 nucleotide (nt) small interfering RNAs (siRNAs) by an RNase III-like enzyme. These siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA. Cleavage of cognate RNA takes place near the middle of the region bound by the siRNA strand.

In this manner, double-stranded RNA (dsRNA) interference may be used. For dsRNA interference, a sense and an antisense RNA molecule that is fully or partially complementary to the sense RNA molecule are expressed in the same cell, resulting in inhibition of the expression of the corresponding endogenous messenger RNA.

The sense and antisense molecules can be expressed from a single or separate expression cassette. Alternatively, multiple plant lines transformed with the dsRNA interference expression cassette or expression cassettes are then screened to identify plant lines that show the greatest inhibition of starch degradation enzyme expression. Methods for using dsRNA interference to inhibit the expression of endogenous plant genes are described in Waterhouse et al. (1998) Proc. Natl. Acad. Sci. USA 95:13959-13964, Liu et al. (2002) Plant Physiol. 129:1732-1743, and WO 99/49029, WO 99/53050, WO 99/61631, and WO 00/49035; each of which is herein incorporated by reference.

In some embodiments of the invention, inhibition of the expression of a starch degradation enzyme may be obtained by hairpin RNA (hpRNA) interference or intron-containing hairpin RNA (ihpRNA) interference. A short hairpin RNA (shpRNA) is a sequence of RNA that makes a tight hairpin turn that can be used to silence gene expression. These methods are highly efficient at inhibiting the expression of endogenous genes. See, Waterhouse and Helliwell (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

Interfering hairpin RNA (ihpRNA) may also be used in the methods of the invention. ihpRNA have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, thus increasing the efficiency of interference. See, for example, Smith et al. (2000) Nature 407:319-320. Methods for using ihpRNA interference to inhibit the expression of endogenous plant genes are described, for example, in Smith et al. (2000) Nature 407:319-320; Wesley et al. (2001) Plant J. 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference. See also WO 02/00904 where the hpRNA is designed such that the loop region determines the specificity of the RNA interference.

In some embodiments of the invention, RNA interference by expression of a gene encoding a micro RNA (miRNA) may be used. miRNAs are regulatory agents consisting of about 22 ribonucleotides. miRNA are highly efficient at inhibiting the expression of endogenous genes. See, for example Javier et al. (2003) Nature 425: 257-263, herein incorporated by reference. For miRNA interference, the expression cassette is designed to express an RNA molecule that is modeled on an endogenous miRNA gene. The miRNA gene encodes an RNA that forms a hairpin structure containing about a 22-nucleotide sequence that is complementary to R1. For example, for suppression of R1 expression, the 22-nucleotide sequence is selected from a starch degradation enzyme transcript sequence and contains 22 nucleotides of said starch degradation enzyme sequence in sense orientation and 21 nucleotides of a corresponding antisense sequence that is complementary to the sense sequence.

Other methods for down-regulating the activity of a target protein include virus-induced gene silencing (Burton et al. (2000) Plant Cell 12:691-705; and Baulcombe (1999) Curr. Op. Plant Bio. 2:109-113); ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); each of which is herein incorporated by reference.

Furthermore, nucleic acid molecules encoding antibodies specifically recognizing a starch degradation enzyme protein, or homologs thereof, according to the invention in a plant cell, i.e. specific fragments or epitopes of such a protein, can be used for inhibiting the activity of this protein. These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Kohler and Milstein (Nature 256 (1975), 495) and Galfre (Meth. Enzymol. 73 (1981) 3), which comprise the fusion of mouse myeloma cells to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. Expression of antibodies or antibody-like molecules in plants can be achieved by methods well known in the art, for example, full-size antibodies (During, Plant. Mol. Biol. 15 (1990), 281-293; Hiatt, Nature 342 (1989), 469-470; Voss, Mol. Breeding 1 (1995), 39-50), Fab-fragments (De Neve, Transgenic Res. 2 (1993), 227-237), scFvs (Owen, Bio/Technology 10 (1992), 790-794; Zimmermann, Mol. Breeding 4 (1998), 369-379; Tavladoraki, Nature 366 (1993), 469-472) and dAbs (Benvenuto, Plant Mol. Biol. 17 (1991), 865-874) have been successfully expressed in tobacco, potato (Schouten, FEBS Lett. 415 (1997), 235-241) or Arabidopsis, reaching expression levels as high as 6.8% of the total protein (Fiedler, Immunotechnology 3 (1997), 205-216).

In addition, nucleic acid molecules encoding a mutant form of the enzyme according to the invention can be used to interfere with the activity of the wild-type protein. Such a mutant form preferably has lost its activity and may be derived from the corresponding wild-type protein by way of amino acid deletion(s), substitution(s), and/or additions in the amino acid sequence of the protein. Mutant forms of such proteins may show, in addition to the loss of activity, an increased substrate affinity and/or an elevated stability in the cell, for instance, due to the incorporation of amino acids that stabilize proteins in the cellular environment. These mutant forms may be naturally occurring or, as preferred, genetically engineered mutants.

It is further contemplated that the methods of the invention can be used with other methods for increasing and/or utilizing the starch content of a plant cob tissue. It is recognized that any mechanism of decreasing the phosphorylation of starch can lead to accumulation of starch in cob tissues, including inhibiting phosphoglucan water dikinase (Kotting et al. (2005) Plant Physiology 137:242-252). Other methods include up-regulating the expression of enzymes involved in the synthesis of starch, for example, ADP-glucose phosphorylase and/or starch synthase.

### Starch Degradation Enzymes' Nucleotide Sequences

The nucleotide sequences for starch degradation enzymes have been identified in Arabidopsis leaves See, alpha-amylase (EC 3.2.1.1), glucan water dikinase (EC 2.7.9.4), Phosphoglucan water dikinase (EC 2.7.9.4), limit dextrinase (EC 3.2.1.142), Isoamylase (EC 3.2.1.68 and EC3.2.1.68), beta-amylase (EC 3.2.1.2), glucan phosphorylase (EC2.4.1.1) and disproportionating enzyme (EC 2.4.1.25). It is recognized that these sequences may be used to down-regulate or suppress expression of the target protein in any plant. However, if additional plant-specific sequence is needed, it can be obtained by hybridization or PCR using the nucleotide sequences noted above.

The nucleotide sequences for R1 proteins from other plants are known in the art. See, for example SEQ ID NO:1 of US Application Publication No. 2006/0282917 (Zea mays); SEQ ID NOs:1, 4, 5, 6, 7, and 9 of U.S. Pat. No. 7,122,727 (wheat); herein incorporated by reference. It is recognized that these sequences may be used to down-regulate or suppress expression of the R1 protein in any plant. However, if additional plant-specific sequence is needed (e.g, an R1 homolog), it can be obtained by hybridization or PCR using sequences based on the R1 nucleotide sequences noted above.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tij ssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

### Plant Expression Cassettes

The compositions of the invention may additionally contain nucleic acid sequences for transformation and expression in a plant of interest. The nucleic acid sequences may be present in DNA constructs or expression cassettes. “Expression cassette” as used herein means a nucleic acid molecule capable of directing expression of a particular nucleotide sequence in an appropriate host cell, comprising a promoter operatively linked to the nucleotide sequence of interest (i.e., an R1 inhibiting polynucleotide) which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. Additionally, the promoter can also be specific to a particular tissue or organ or stage of development.

The present invention encompasses the transformation of plants with expression cassettes capable of expressing polynucleotides that reduce or eliminate the activity of one or more starch degradation enzymes. The expression cassette will include in the 5′-3′ direction of transcription, a transcriptional and translational initiation region (i.e., a promoter) and a polynucleotide of interest, i.e., a polynucleotide capable of directly or indirectly (i.e. via expression of a protein product) reducing or eliminating the activity of one or more starch degradation enzymes. The expression cassette may optionally comprise a transcriptional and translational termination region (i.e. termination region) functional in plants. In some embodiments, the expression cassette comprises a selectable marker gene to allow for selection for stable transformants. Expression constructs of the invention may also comprise a leader sequence and/or a sequence allowing for inducible expression of the polynucleotide of interest. See, Guo et al. (2003) Plant J. 34:383-92 and Chen et al. (2003) Plant J. 36:731-40 for examples of sequences allowing for inducible expression.

The regulatory sequences of the expression construct are operably linked to the polynucleotide of interest. By “operably linked” is intended a functional linkage between a promoter and a second sequence wherein the promoter sequence initiates and mediates transcription of the DNA sequence corresponding to the second sequence. Generally, operably linked means that the nucleotide sequences being linked are contiguous.

Any promoter capable of driving expression in the plant of interest may be used in the practice of the invention. The promoter may be native or analogous or foreign or heterologous to the plant host. The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA or RNA) sequence naturally associated with a host cell into which it is introduced.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence.

Some suitable promoters initiate transcription only, or predominantly, in certain cell types. Thus, as used herein a cell type- or tissue-preferential promoter is one that drives expression preferentially in the target tissue, but may also lead to some expression in other cell types or tissues as well. It is understood that some promoters that show preferential targeting of expression in target tissues may also exhibit “leaky” expression in non-preferential targeted tissues. One example may be a promoter whose expression profile shows preferential expression in maize seed however also exhibits strong expression in mature leaf tissue. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Promoters showing preferred activity in photosynthetic tissue with some activity in cob tissue may be useful in some embodiments of the invention. The promoter may confer expression constitutively throughout the plant, or differentially with respect to the plant cob tissues, or differentially with respect to the developmental stage of the plant cob tissue in which expression occurs, or in response to external stimuli.

Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant Cell Physiol. 35:773-778), the Cab-1 gene promoter from wheat (Fejes et al. (1990) Plant Mol. Biol. 15:921-932), the CAB-1 promoter from spinach (Lubberstedt et al. (1994) Plant Physiol. 104:997-1006), the cablR promoter from rice (Luan et al. (1992) Plant Cell 4:971-981), the pyruvate orthophosphate dikinase (PPDK) promoter from maize (Matsuoka et al. (1993) Proc Natl Acad Sci USA 90:9586-9590), the tobacco Lhcb1*2 promoter (Cerdan et al. (1997) Plant Mol. Biol. 33:245-255), the Arabidopsis thaliana SUC2 sucrose-H+ symporter promoter (Truernit et al. (1995) Planta 196:564-570), and thylakoid membrane protein promoters from spinach (psaD, psaF, psaE, PC, FNR, atpC, atpD, cab, rbcS. Other promoters that drive transcription in stems, leafs and green tissue are described in U.S. Patent Publication No. 2007/0006346, herein incorporated by reference in its entirety.

In some other embodiments of the present invention, inducible promoters may be desired. Inducible promoters drive transcription in response to external stimuli such as chemical agents or environmental stimuli. For example, inducible promoters can confer transcription in response to hormones such as gibberellic acid or ethylene, or in response to light or drought. Senescent inducible promoters may also be used in the current invention to suppress starch degradation enzymes at specific developmental stages of the plant so that starch accumulates in plant cob tissues at a specific time. One example would be to inhibit or suppress the starch degradation enzyme in maize cob following the maturation of seed endosperm. Some other examples of inducible promoters that may be used in various embodiments of the invention may be found in Journal of Experimental Botany 2008 59(2):377-387; Senescence-induced ectopic expression of the A. tumefaciens ipt gene in wheat delays leaf senescence, increases cytokinin content, nitrate influx, and nitrate reductase activity, but does not affect grain yield, Blanka S\grave{y}ikorová1, Gabriela Kure\check{s}ová2, Sasha Daskalova3,*, Marie Tr\check{c}ková2, Klára Hoyerová1, Ivana Raimanová2, Václav Motykal, Alena Trávní\check{c}ovál, Malcolm C. Elliott3 and Miroslav Kamínek1,† PNAS Dec. 4, 2007 vol. 104 no. 49 19631-19636 Delayed leaf senescence induces extreme drought tolerance in a flowering plant, and Rosa M. Rivero*, Mikiko Kojima†, Amira Gepstein‡, Hitoshi Sakakibara†, Ron Mittler§¶, Shimon Gepstein†, and Eduardo Blumwald, Plant Physiol. (1999) 120: 1015-1024 all herein incorporated by reference.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used.

Generally, the expression cassette will comprise a selectable marker gene for the selection of transformed cells. Selectable marker genes are utilized for the selection of transformed cells or tissues.

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picomavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, the section entitled “Expression With Chloroplast Targeting” in Example 37 of U.S. Pat. No. 5,639,949.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

In order to ensure the localization in the plastids it is conceivable to use one of the following transit peptides: of the plastidic Ferredoxin: NADP+ oxidoreductase (FNR) of spinach which is enclosed in Jansen et al. (Current Genetics 13 (1988), 517-522). In particular, the sequence ranging from the nucleotides −171 to 165 of the cDNA sequence disclosed therein can be used, which comprises the 5′ non-translated region as well as the sequence encoding the transit peptide. Another example is the transit peptide of the waxy protein of maize including the first 34 amino acid residues of the mature waxy protein (Klosgen et al., Mol. Gen. Genet. 217 (1989), 155-161). It is also possible to use this transit peptide without the first 34 amino acids of the mature protein. Furthermore, the signal peptides of the ribulose bisposphate carboxylase small subunit (Wolter et al., Proc. Natl. Acad. Sci. USA 85 (1988), 846-850; Nawrath et al., Proc. Natl. Acad. Sci. USA 91 (1994), 12760-12764), of the NADP malate dehydrogenase (Galiardo et al., Planta 197 (1995), 324-332), of the glutathione reductase (Creissen et al., Plant J. 8 (1995), 167-175) or of the R1 protein Lorberth et al. (Nature Biotechnology 16, (1998), 473-477) can be used.

### Plant Transformation

Once a starch degradation enzyme inhibiting nucleic acid sequence has been cloned into an expression system, it is transformed into a plant cell. The receptor and target expression cassettes of the present invention can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, herein incorporated by reference.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders. Agrobacterium mediated In Planta transformation system is also used routinely for Arabidopsis (Bechtold et. al. CR Acad. Sci III. Sci Vie 316:1194-1199 (1993) and can be used for Brassica napus (Wang W. C. et. al. Plant Cell Report 22:274-281 (2003)

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11:1553-1558 (1993)) and Weeks et al. (Plant Physiol. 102: 1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For example, rice (Oryza sativa) can be used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 gaiter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about two days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., (2002) In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T₁ seed is harvested.

The plants obtained via transformation with a nucleic acid sequence of the present invention can be any of a wide variety of plant species; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

For the transformation of plastids, seeds of Nicotiana tabacum c.v. “Xanthienc” are germinated seven per plate in a 1″ circular array on T agar medium and bombarded 12-14 days after sowing with 1 um tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 umol photons/m²/s) on plates of RMOP medium (Svab, Z., Hajdukiewicz, P. and Maliga, P. (1990) PNAS 87, 8526-8530) containing 500 ug/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with .sup.32P-labeled random primed DNA sequences corresponding to a 0.7 kb BamHI/HindIII DNA fragment from pC8 containing a portion of the rps 7/12plastid targeting sequence. Homoplasmic shoots are rooted aseptically on spectinomycin-containing MS/IBA medium (McBride, K. E. et al. (1994) PNAS 91, 7301-7305) and transferred to the greenhouse.

The genetic properties engineered into the transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the transgenic plants and seeds according to the invention can further be made in plant breeding. Depending on the desired properties, different breeding measures are taken. The relevant techniques are well known in the art and include but are not limited to hybridization, inbreeding, double haploid, backcross breeding, multi-line breeding, variety blend, interspecific hybridization, aneuploid techniques, etc. Thus, the transgenic seeds and plants according to the invention can be used for the breeding of improved plant lines that, for example, increase the effectiveness of conventional methods such as herbicide or pesticide treatment or allow one to dispense with said methods due to their modified genetic properties.

### Biomass Conversion

Plants transformed according to the present invention provide a means of increasing ethanol yields, reducing pretreatment costs, by reducing acid/heat pretreatment requirements for saccharification of biomass; and/or reducing other plant production and processing costs, such as by allowing multi-applications and isolation of commercially valuable by-products

Harvesting methods of collecting cob has been recently developed in the art. For example, whole cob plus kernel is collected by a modified combine. The combine may be modified to both harvest cob and further strip and store cob and seed in separate bins within the combine. Another method may be for the combine to collect whole cob plus kernel and then separate kernel and cob at the ethanol production facility. Another option might be to use a conventional combine that discharges stripped cob into an accompanying truck bed. Full cob may be stripped of the kernel at the biomass conversion facility where the corn seed in one stream is fractionated into its' relative components (e.g., endosperm, fiber, germ). Afterwards, these corn seed components may be further processed to produce commercial products. For example the fractionated corn seed endosperm may be utilized in a commercial yeast fermentation to produce ethanol. The cob may be directed to a second stream where it may or may not be pretreated and then fermented to produce a favorable commercial product (e.g., ethanol, butanol). Another method may be to ferment whole cob plus kernel in a single batch. An embodiment of the following invention may be to use a cob with increased starch in any of the methods mentioned above. Another embodiment may be to enable conversion of whole cob plus kernel to a commercial product (e.g., ethanol) through a single batch process where the whole cob with increased levels of starch and kernel is ground and further sacchrafied and fermented in a biomass conversion. Another embodiment may be to process whole cob with increased levels of starch and kernel to be used in animal feed. Another embodiment may be to harvest entire above-the-ground plant parts containing an increased level of starch, cob containing an increased level of starch and corn seed to be further utilized in a biomass conversion method. In one embodiment it may be desirable to practice the invention in a multi-eared corn variety. In yet another embodiment, cob with increased starch content may be used as a tissue to express large amounts of enzyme for the production of commercial enzymes. For example, expression of a cellulase or amylase might be expressed at high levels in the cob containing elevated amounts of starch followed by processing to extract the enzyme for commercial distribution from said cob. In addition, the starch may add additional value in a biomass conversion application.

Pretreatment.

Conventional methods include physical, chemical, and/or biological pretreaments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol. Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society: B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes. One embodiment of the application may be to shorten or rid of the cob pretreatment step by using a cob with increased starch levels. Another embodiment may be to express a processing enzyme in cob or seed that would further breakdown complex molecules into simple sugars to be used in a biomass conversion method. Another embodiment may be to express a processing enzyme in cob or seed that pretreat complex molecules for quicker conversion into a fermentable sugar to be used in a biomass conversion method. For example, one may engineer a transgenic maize plant wherein the cob of the transgenic maize plant has an increased level of starch by the downregulation of a starch degradation enzyme, the transgenic plant is further engineered to express a processing enzyme (e.g., alpha-amylase, cellulase) in the cob or corn seed where either whole cob plus seed or seed separated from cob are fermented essentially as described above. The processing enzyme may be activated upon milling and processing conditions. Another embodiment may be to engineer a low lignin cob as described in U.S. 2006/0260011 herein incorporated by reference, to have an increased level of starch. A low lignin cob with elevated amounts of starch may be useful in silage, biomass conversion methods and in animal feed.

Saccharification.

In saccharification (or enzymatic hydrolysis), lignocellulose is converted into fermentable sugars by lignocellulolytic enzymes present in the pretreated material or exogenously added. Saccharification is generally performed in stirred-tank reactors or fermentors under controlled pH, temperature, and mixing conditions. A saccharification step may last up to 200 hours. Saccharification may be carried out at temperatures from about 30.degree. C. to about 65.degree. C., in particular around 50.degree. C., and at a pH in the range of between about 4 and about 5, in particular, around pH 4.5. Saccharification can be performed on the whole pretreated material. An embodiment of the application may be to further engineer a cob with increased starch to express one or more processing enzymes to speed up saccharification or to make the process more efficient. Another embodiment may be in a process using whole cob with increased starch content plus intact kernel to express one or more processing enzymes in the corn seed and perform whole cob plus kernel saccharification. The processing enzyme may be activated upon milling and processing conditions.

Fermentation.

In the fermentation step, sugars, released from the lignocellulose as a result of the pretreatment and enzymatic hydrolysis steps, are fermented to one or more organic substances, e.g., ethanol, by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF. SSF for C6 sugar, SSCF or simultaneous saccharification and cofermentation for C5 and C6 sugar may be combined.

Fermenting microorganisms and methods for their use in ethanol production are known in the art (Sheehan, “The road to Bioethanol: A strategic Perspective of the US Department of Energy's National Ethanol Program” In: “Glucosyl Hydrolases For Biomass Conversion”, ACS Symposium Series 769, 2001, American Chemical Society: Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Yeast or other microorganisms are typically added to the hydrolysate and the fermentation is allowed to proceed for 24-96 hours, such as 35-60 hours. The temperature of fermentation is typically between 26-40.degree. C., such as 32.degree. C., and at a pH between 3 and 6, such as about pH 4-5.

A fermentation stimulator may be used to further improve the fermentation process, in particular, the performance of the fermenting microorganism, such as, rate enhancement and ethanol yield. Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

Recovery.

Following fermentation (or SSF), the mash is distilled to extract the ethanol. Ethanol with a purity greater than 96 vol. % can be obtained.

Combined Starch Hydrolysis and Cellulolytic Material Hydrolysis.

The transgenic plants and plant parts disclosed herein can be used in methods involving combined hydrolysis of starch and of cellulosic material for increased ethanol yields. In addition to providing enhanced yields of ethanol, these methods can be performed in existing starch-based ethanol processing facilities.

Starch is a glucose polymer that is easily hydrolyzed to individual glucose molecules for fermentation. Starch hydrolysis may be performed in the presence of an amylolytic microorganism or enzymes such as amylase enzymes. In certain embodiments of the invention, starch hydrolysis is performed in the presence of at least one amylase enzyme. Examples of suitable amylase enzymes include alpha-amylase (which randomly cleaves the alpha(1-4)glycosidic linkages of amylose to yield dextrin, maltose or glucose molecules) and glucoamylase (which cleaves the .alpha.(1-4) and .alpha.(1-6)glycosidic linkages of amylose and amylopectin to yield glucose).

In the inventive methods, hydrolysis of starch and hydrolysis of cellulosic material can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate lignocellulolytic enzyme(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

The methods may use transgenic plants (or plant parts) alone or a mixture of non-transgenic plants (or plant parts) and plants (or plant parts) transformed according to the present invention. Suitable plants include any plants that can be employed in starch-based ethanol production (e.g., corn). For example, the present inventive methods may be used to increase ethanol yields from corn cob.

The plants of the invention find use in biomass conversion methods for producing sugars or biofuels from plant biomass. Herein, the term “biofuels” refers to any fuel derived from harvested plant parts. Biofuels comprise but are not limited to biodiesel, vegetable oils, bioalcohols (i.e. ethanol, methanol, propanol, butanol, etc.) and biogases (i.e. methane). The plants of the invention are engineered to accumulate higher concentrations of starch in their cob tissues thus providing a rich source of carbohydrates which then can be converted to biofuels. Herein, the term “free sugars” defines any carbohydrate derived from plant biomass that can be further processed to make fermentable sugars, chemicals, biofuels, plastics, feed additives or any other commercially important product. One embodiment of the current application provides a method of improving the yield of free sugars from plant cob biomass comprising manipulating a plant to down-regulate the activity of one or more starch degradation enzymes in cob. The resultant plant cob will contain increased levels of starch which then can be converted to free sugars in a conventional biomass conversion method. Herein, the term “biomass conversion method” defines any process that converts plant parts into fermentable sugars, biofuels, chemicals, plastics, feed additives, or any other commercially important products. Biomass conversion methods may also contain a subcategory herein referred to as a “non-animal feed biomass conversion method”. Non-animal feed biomass conversion method defines any process that converts plant parts into fermentable sugars, biofuels, chemicals and plastics not destined for animal consumption.

The compositions and methods of the invention are useful in the production of dextrose for fructose syrups, specialty sugars, and in alcohol and other end-product (e.g. organic acid, ascorbic acid, and amino acids) production from fermentation of starch (G. M. A van Beynum et al., Eds. (1985) Starch Conversion Technology, Marcel Dekker Inc. NY). Production of alcohol from the fermentation of starch derived from the cob tissues of the plants of the invention may include the production of fuel alcohol or potable alcohol.

In certain preferred embodiments, the alcohol will be ethanol. In particular, alcohol fermentation production processes are characterized as wet milling or dry milling processes. In some embodiments, the plants are subjected to a wet milling fermentation process and, in other embodiments, a dry milling process is used. In certain embodiments, ethanol may be produced using a raw starch hydrolysis method. Another embodiment may be whole cob with increased starch content plus kernel utilized in a raw starch hydrolysis. Another embodiment may be the addition of one or more processing enzymes in a raw starch hydrolysis containing whole cob with increased starch content and kernel. In other embodiments ground cob with increased starch content expressing a processing enzyme of interest may be ground and sold as a additive for either animal feed or in use for a biomass conversion method.

Dry grain milling involves a number of basic steps, which generally include: grinding, cooking, liquefaction, saccharification, fermentation and separation of liquid and solids to produce alcohol and other co-products. Plant material and particularly whole cereal grains, such as maize, sorghum, wheat or rye are ground. In some cases the grain may be first fractionated into component parts. The ground plant material may be milled to obtain a coarse or fine particle. The ground plant material is mixed with liquid in a slurry tank. The slurry is subjected to high temperatures in a jet cooker along with liquefying enzymes (e.g. alpha amylases) to solubles and hydrolyze the starch in the cereal to dextrins. The mixture is cooled down and further treated with saccharifying enzymes to produce glucose. The mash containing glucose is then fermented for approximately 24 to 120 hours in the presence of fermentation microorganisms, such as ethanol producing microorganism and particularly yeast (Saccharomyces spp). The solids in the mash are separated from the liquid phase and alcohol such as ethanol and useful co-products such as distillers' grains are obtained. In one embodiment the addition of a cob with increased starch to a dry mill facility could increase the quality of DDGS in regards to animal feed and available nutrients.

In some embodiments, the saccharification step and fermentation step are combined and the process is referred to as simultaneous saccharification and fermentation or simultaneous saccharification, yeast propagation and fermentation.

In other embodiments, the cooking step or exposure of the cob starch containing substrate to temperatures above the gelatinization temperate of the starch in the substrate may be eliminated. These fermentation processes in some embodiments include milling of a cereal grain or fractionated grain and combining the ground cereal grain with liquid to form a slurry, which is then mixed in a single vessel with amylases, glucoamylases, and/or other enzymes having granular starch hydrolyzing activity and yeast to produce ethanol and other co-products (U.S. Pat. No. 4,514,496, WO 04/081193 and WO 04/080923). In some embodiments, the enzymes useful for fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

In another embodiment, the invention is directed to a transformed plant, the genome of which is augmented with a recombinant polynucleotide encoding at least one processing enzyme operably linked to a promoter sequence, the sequence of which polynucleotide is optimized for expression in the plant. It may be beneficial to create a plant with increased cob starch that has been further modified to express a processing enzyme that when activated will be capable of self-processing the substrate upon which it acts to obtain the desired result as described in, US20030135885 and U.S. Pat. No. 7,102,057 herein incorporated by reference. Herein a cob with increased starch and further modified with a processing enzyme is referred to as a “self-processing cob with increased starch content”. Provided are methods of producing a self-processing cob with increased starch content wherein the plant or plant part expresses a processing enzyme (e.g., alpha-amylase, glucoamylase, cellulases, CBHI, etc.) wherein upon activation (e.g. milling, addition of water, pH) of the processing enzyme(s) (mesophilic, thermophilic, or hyperthermophilic) the plant or plant part is capable of self-processing the substrate upon which it acts to obtain the desired result. In some embodiments the processing enzyme may be expressed in other plant parts (e.g. seed or green tissue) of the maize plant with increased cob starch wherein said other plant parts are processed along with cob with increased starch thus bringing the enzyme in contact with the cob starch upon processing. In another embodiment it may be desired to express one or more processing enzymes in one crop plant and downregulate a starch degradation enzyme in a maize plant to produce a cob with increased starch content. These feedstocks could then be mixed in a biomass conversion method wherein the processing enzymes will come in contact with their respective substrate and activated upon processing for example milling and mixing the two feedstocks into a single liquefaction.

In accordance with the present invention, a “self-processing” plant or plant part has incorporated therein an isolated polynucleotide encoding a processing enzyme capable of processing, e.g., modifying, starches, polysaccharides, lipids, proteins, and the like in plants, wherein the processing enzyme can be mesophilic, thermophilic or hyperthermophilic, and may be activated by grinding, addition of water, heating, or otherwise providing favorable conditions for function of the enzyme. The isolated polynucleotide encoding the processing enzyme is integrated into a plant or plant part for expression therein. Upon expression and activation of the processing enzyme, the plant or plant part of the present invention processes the substrate upon which the processing enzyme acts. Therefore, the plant or plant parts of the present invention are capable of self-processing the substrate of the enzyme upon activation of the processing enzyme contained therein in the absence of or with reduced external sources normally required for processing these substrates. As such, the transformed plants, transformed plant cells, and transformed plant parts have “built-in” processing capabilities to process desired substrates via the enzymes incorporated therein according to this invention. Preferably, the processing enzyme-encoding polynucleotide are “genetically stable,” i.e., the polynucleotide is stably maintained in the transformed plant or plant parts of the present invention and stably inherited by progeny through successive generations.

In accordance with the present invention, methods which employ such plants and plant parts can eliminate the need to mill or otherwise physically disrupt the integrity of plant parts prior to recovery of starch-derived products. For example, the invention provides improved methods for processing cob to recover starch-derived products. The invention also provides a method which allows for the recovery of starch granules that contain levels of starch degrading enzymes, in or on the granules that are adequate for the hydrolysis of specific bonds within the starch without the requirement for adding exogenously produced starch hydrolyzing enzymes. The invention also provides improved products from the self-processing plant or plant parts obtained by the methods of the invention.

In addition, the “self-processing” transformed plant part, e.g., cob, and transformed plant avoid major problems with existing technology, i.e., processing enzymes are typically produced by fermentation of microbes, which requires isolating the enzymes from the culture supernatants, which costs money; the isolated enzyme needs to be formulated for the particular application, and processes and machinery for adding, mixing and reacting the enzyme with its substrate must be developed. The transformed plant of the invention or a part thereof is also a source of the processing enzyme itself as well as substrates and products of that enzyme, such as sugars, amino acids, fatty acids and starch and non-starch polysaccharides. The plant of the invention may also be employed to prepare progeny plants such as hybrids and inbreds.

The plant may be a monocot, such as maize. Preferably, the plant is a energy crop or a commercially grown plant. Herein the term “processing enzyme” is selected from the group consisting of an α-amylase, glucoamylase, glucose isomerase, glucanase, β-amylase, α-glucosidase, isoamylase, pullulanase, neo-pullulanase, iso-pullulanase, amylopullulanase, cellulase, exo-1,4-β-cellobiohydrolase, exo-1,3-β-D-glucanase, β-glucosidase, endoglucanase, L-arabinase, α-arabinosidase, galactanase, galactosidase, mannanase, mannosidase, xylanase, xylosidase, protease, glucanase, esterase, phytase, and lipase. Preferably, the processing enzyme is a starch-processing enzyme selected from the group consisting of α-amylase, glucoamylase, glucose isomerase, β-amylase, α-glucosidase, isoamylase, pullulanase, neo-pullulanase, iso-pullulanase, and amylopullulanase. More preferably, the enzyme is selected from α-amylase, cellulase, glucoamylase, glucose isomerase, glucose isomerase, α-glucosidase, and pullulanase. The processing enzyme is further preferably hyperthermophilic. In accordance with this aspect of the invention, the enzyme may be a non-starch degrading enzyme selected from the group consisting of protease, glucanase, xylanase, esterase, phytase, and lipase. Such enzymes may further be hyperthermophilic. In a preferred embodiment, the enzyme accumulates in the vacuole, endoplasmic reticulum, apoplast, protein storage vacuole, mitochondria, chloroplast, starch granule, seed or cell wall of a plant. Moreover, in another embodiment, the genome of plant may be further augmented with a second recombinant polynucleotide comprising a non-hyperthermophilic enzyme. In another embodiment it may be desired to increase the starch granule size in a cob with increased starch by the downregulation of starch synthase IV and/or starch phosphorylase using methods such as those referenced in WO 2005/097999. In another embodiment it may be desired to modify the cob starch to produce unique sugar profiles.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXAMPLES

The invention will be further described by the following examples, which are not intended to limit the scope of the invention in any manner.

### Example 1

### Generation of Transgenic Maize Plants

Eight RNAi cassettes were constructed for plant expression. These cassettes were designed to down-regulate the enzymes α-amylase, β-amylase, glucan water dikinase (R1), phosphoglucan water dikinase (PWD), and a chloroplastic α-amylase (AMY3) in maize cob tissue. The Zea mays cDNA sequences for α-amylase (Genbank Accession L25805), β-amylase (Genbank Accession Z25871) and α-glucan water dikinase (R1, Genbank Accession CD973834) were obtained from NCBI. A maize ortholog for Arabidopsis thaliana's chloroplastidic phosphoglucan water dikinase (GenBank Accession AJ635427) was generated based on sequence homology against maize genomic sequence. Likewise, a maize ortholog for the Arabidopsis thaliana Amy3 (GenBank Accession NM105651) was generated based on sequence homology against maize genomic sequence. RNAi fragments (495 bp of α-amylase, SEQ ID NO: 1; 500 bp of β-amylase, SEQ ID NO: 2; 330 bp of R1, SEQ ID NO: 3, 320 bp of PWD, SEQ ID NO: 4; and 320 bp of AMY3, SEQ ID NO: 5) from the 3′-ends of the coding regions of the respective genes were synthesized by Geneart (Geneart AG). During synthesis, attB1 and attB2 sites were added to 5′ and 3′ ends of these eight RNAi sequences, respectively. In addition an intron from the rice sucrose synthase-1 (RSsl) gene was synthesized on the corresponding strand relative to the RNAi sequence to act as the spacer in the hair-pin loop formed by pairing the corresponding RNAi fragments, thus providing a binding region for the enzyme dicer. All eight RNAi expression cassettes were recombined into pDONR221 using the BP Reaction from Gateway® Cloning Technology (Invitrogen Life Science).

Destination vector 15910 is a binary vector containing a phosphomannose isomerase (PMI) gene that allows selection of transgenic cells with mannose. Vector 15910 also provides a method for making an RNAi cassette using Gateway® Cloning Technology (Invitrogen Life Science). Destination vector 15910 in addition contains a double transcriptional enhancer (Figwort mosaic virus (FMV) enhancer) as well as a Cauliflower mosaic virus 35s enhancer. Vector 15910 allows for a means to change in an out various promoters which drive expression of the RNAi cassettes in maize cob tissue. Promoters used in this study were the Oryza sativa MADS-box 13 (OsMADS 13) promoter, the maize tryptophan synthase alpha subunit (TrpA) promoter, the MADS-box 14 (OsMADS14) promoter and the maize Zm015970 promoter. The Oryza sativa MADS-box 13 (OsMADS 13) promoter which is further described in publication US 2007/0006344 and depicted in SEQ ID NO: 6 has been shown to preferably express genes in maize cob tissue. The maize tryptophan synthase alpha subunit (TrpA) promoter as described in Plant Mol Bio (1995) 27:1183-1188 and as depicted in SEQ ID NO: 7 is expected to drive expression preferably in the pith and young leaves of maize plants. The OsMADS14 promoter as described in publication U.S. 2007/0006344 and depicted in SEQ I.D. NO: 9 preferably drives the expression of transgenes in maize cob tissue. The maize promoter Zm015970 as depicted in SEQ ID NO: 8 was identified to be a strong cob-preferred promoter based on maize microarray data. The ratio of the level of expression of a gene driven by Zm015970 in corn cob compared to non-cob tissue was approximately 7.9. The promoter sequence of the Zm015970 is composed of 2009 bp of the 5′UTR, 510 bp of the first exon of the gene, 132 bp of the first intron, and 19 bp of the second exon of the Zm015970 genomic clone.

The Gateway Entry RNAi cassettes (SEQ ID #s 10-17) created by Geneart were recombined into vector 15910 using the LR Reaction (Gateway® LR Clonase® Enzyme Mix, Invitrogen), creating binary vectors. Promoters were Binary vectors were verified by restriction digest and sequencing.

Transformation of immature maize embryos is performed essentially as described in Negrotto et al., Plant Cell Reports 19:798-803 (2000). Various media constituents described therein can be substituted.

Agrobacterium strain LBA4404 (Invitrogen) containing the plant transformation plasmid is grown on YEP (yeast extract (5 g/L), peptone (10 g/L), NaCl (5 g/L), 15 g/l agar, pH 6.8) solid medium for 2 to 4 days at 28° C. Approximately 0.8×109 Agrobacteria are suspended in LS-inf media supplemented with 100 μM acetosyringone (As) (LSAs medium) (Negrotto et al., Plant Cell Rep 19:798-803 (2000)). Bacteria are pre-induced in this medium for 30-60 minutes.

Immature embryos from maize line, A188, or other suitable maize genotypes are excised from 8-12 day old ears into liquid LS-inf+100 μM As (LSAs). Embryos are vortexed for 5 seconds and rinsed once with fresh infection medium. Infection media is removed and Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/1) and silver nitrate (1.6 mg/1) (Negrotto et al., Plant Cell Rep 19:798-803 (2000)) and cultured in the dark for 28° C. for 10 days.

Immature embryos producing embryogenic callus are transferred to LSD1M0.5S medium (LSDc with 0.5 mg/l 2,4-D instead of Dicamba, 10 g/l mannose, 5 g/l sucrose and no silver nitrate). The cultures are selected on this medium for 6 weeks with a subculture step at 3 weeks. Surviving calli are transferred either to LSD1M0.5S medium to be bulked-up or to Reg1 medium (as described in Negrotto et al., Plant Cell Rep 19:798-803 (2000)). Following culturing in the light (16 hour light/8 hour dark regiment), green tissues are then transferred to Reg2 medium without growth regulators (as described in Negrotto et al., Plant Cell Rep 19:798-803 (2000)) and incubated for 1-2 weeks. Plantlets are transferred to Magenta GA-7 boxes (Magenta Corp, Chicago Ill.) containing Reg3 medium (as described in Negrotto et al. (2000)) and grown in the light. Plants that were PCR positive for PMI and iOsSH1 i1-01 (loop) and negative for Spectinomycin were transferred to soil and grown in the greenhouse. A total of 267 T⁰ plants were grown to maturity (Table 2). Plant samples of selected events were collected for Lugol's staining, starch analysis and fermentation analysis.

TABLE 1
Constructs for Expression of RNAi Cassettes in Maize Cob Tissue

| Construct | Expression Cassette (promoter & gene) |
|---|---|
| 17303 | OsMADS13 + alpha-amylase |
| 17304 | OsMADS13 + beta-amylase |
| 17305 | OsMADS13 + R1 |
| 18278 | OsMADS13 + PWD |
| 18222 | OsMADS13 + AMY3 |
| 18286 | TrpA + R1 |
| 18364 | OsMADS14 + R1 |
| 18288 | Zm015970 + R1 |

TABLE 2
Number of T⁰ events carried forward to the greenhouse to maturity.

| | Number of T⁰ Transferred to |
|---|---|
| Construct | Greenhouse |
| 17303 | 40 (38L + 2M) |
| 17304 | 39 (38L + 1M) |
| 17305 | 37 (26L + 11M) |
| 18278 | 29 (4N + 10L + 7M + 8H) |
| 18222 | 50 (5N + 18L + 16M + 11H) |
| 18286 | 44 (7N + 28L + 9M) |
| 18288 | 28 (3N + 6L + 7M + 12H) |

*Taqman ® PCR copy number, N—nulls, L—low copy, M—Medium copy, H—High copy

### Example 2

### Sample Collection and Prescreening of Transgenic Maize Events

Cob samples were taken from mature T⁰ maize plants 10 days post-pollination. Fresh cobs were then frozen at −80° C. Following, seeds were removed from frozen cob using a paint scrapper. Freezing the cob allows for better removal of corn seeds for cob analysis. After removal of cob seed, each cob was then sliced less than ¼inch thick. Two of the slices (one from the middle and one from the end) were used for Lugol's iodine staining Control plants can be generated by using T⁰ plants transformed with empty binary vectors containing no RNAi cassettes using the methods as described in Example 1.

Prescreening of starch accumulation in T⁰ maize events was carried out using Lugol's staining solution. Lugol's solution selectively stains starch dark blue to black, and can be observed visually under a microscope. The degree of staining relative to null controls allowed for the fast visual preselection of events with increased starch in cob tissues to be carried forward for further starch analysis and fermentation studies.

Prescreening of event cob tissue was carried out by placing the cob sections in a 50-mL conical centrifuge tube and adding 5 mL of 5% Lugol's Iodine solution to each sample making sure the entire sample in covered. Samples were then stained for three hours at room temperature. The Lugol's solution was then removed and samples were washed with distilled water by allowing the samples to sit in water, changing the water often until no more stain could be seen in the wash water. Cob cross sections were placed on a microscope slide and allowed to dry prior to scoring events. Once dried, cob samples were visualized on a white background and visually scored against control sample for amount of starch staining Scoring was carried out by assigning a numerical value to each sample (1-5, where 1=low starch accumulation, 5=high starch accumulation) based on stain intensity which reflected the starch content/accumulation; and recorded photographically. Relative Lugol staining scores are outlined in Tables 3-9. Visual scores were used to determine which events to carry out further starch analysis and fermentation experiments. It was surprisingly found in the lab that starch accumulation in cob tissue of maize plants as indicated by starch staining, was significantly increased in maize plants where alpha-amylase, beta-amylase, R1, PWD, or AMY3 have been downregulated in cob tissues comparative to control cob.

TABLE 3
Construct 17303 Cob Starch Lugol's Staining (OsMADS13
promoter + alpha-amylase RNAi)

| Plant ID | Lugol's Staining Rating |
|---|---|
| Control 1 | 1 |
| Control 2 | 1 |
| MZBF080319A015A | 3 |
| MZBF080319A027A | 1-2 |
| MZBF080319A040A | 3-4 |
| MZBF080319A052A | 3-4 |
| MZBF080319A068A | 3 |

TABLE 4
Construct 17304 Cob Starch Lugol's Staining (OsMADS13
promoter + beta-amylase RNAi)

| Plant ID | Lugol's Staining Rating |
|---|---|
| Control 1 | 1 |
| Control 2 | 1 |
| MZBF080321A001A | 3-4 |
| MZBF080321A010A | 2-3 |
| MZBF080321A013A | 2-3 |
| MZBF080321A014A | 2-3 |
| MZBF080321A030A | 1 |

TABLE 5
Construct 17305 Cob Starch Lugol's Staining
(OsMADS13 promoter + glucan water dikinase (R1) RNAi)

| Plant ID | Lugol's Staining Rating |
|---|---|
| Control 1 | 1 |
| Control 2 | 1 |
| MZBF080322A007A | 5 |
| MZBF080322A013A | 2-3 |
| MZBF080322A035A | 5 |
| MZBF080322A038A | 5 |
| MZBF080322A072A | 5 |

TABLE 6
Construct 18278 Cob Starch Lugol's Staining
(OsMADS13 promoter + PWD RNAi)

| Plant ID | Lugol's Staining Rating |
|---|---|
| Control | 2 |
| MZBF093523A048A | 3 |
| MZBF093523B049A | 2 |
| MZBF093523A045A | 2 |
| MZBF093523B004A | 2 |
| MZBF093523A051A | 3 |

TABLE 7
Construct 18222 Cob Starch Lugol's Staining
(OsMADS13 promoter + AMY3 RNAi)

| Plant ID | Lugol's Staining Rating |
|---|---|
| Control | 2 |
| MZBF093606A040A | 3 |
| MZBF093606B045A | 3 |
| MZBF093606B008A | 3 |
| MZBF093606A054A | 3 |
| MZBF093606A005A | 3 |

TABLE 8
Construct 18286 Cob Starch Lugol's Staining
(TrpA promoter + R1 RNAi)

| Plant ID | Lugol's Staining Rating |
|---|---|
| Control | 2 |
| MZBF093527A043B | 4 |
| MZBF093527A005A | 4 |
| MZBF093527A030A | 4 |
| MZBF093527A013A | 5 |
| MZBF093527C032A | 5 |

TABLE 9
Construct 18288 Cob Starch Lugol's Staining
(Zm015970 promoter + R1 RNAi)

| Plant ID | Lugol's Staining Rating |
|---|---|
| Control | 2 |
| MZBF093516C003A | 3 |
| MZBF093516C002A | 3 |
| MZBF093516B006A | 3 |
| MZBF093516B010A | 3 |
| MZBF093516A005A | 4 |

### Example 3

### Starch and Sugar Estimation in Maize Cob Tissue Samples

The following assay procedure is used to estimate the amount of sugar and total starch in cob samples on a dry weight basis. This method employs the Megazyme Total Starch Assay (MEGAZYME, Wicklow, Ireland) (AOAC Method 996.11 and AACC Method 76.13) which involves complete digestion of sample starch to free D-glucose by an alpha-amylase and an amyloglucosidase hydrolysis followed by a glucose oxidase-peroxidase reaction and colorimetric measurement of free D-glucose liberated from the sample. The amount of starch in the sample can be calculated through a simple conversion of the amount of released D-glucose measurement. The sugars are extracted with water and the amount is analyzed by HPAEC (High performance Anion Exchange Chromatography).

### Cob Sample Preparation

Fresh cobs were frozen at 80° C., photographed, and the seeds were removed using a paint scrapper. Each cob was then sliced into slices less than ¼inch thick. Slices for each cob were placed in a weigh boat and dried using a lyophilizer. Two of the slices (one from the middle and one from the end) were put aside for Lugol's Iodine staining (as depicted in Example 2). The remaining cob slices were ground using a centrifuge mill. To achieve the required grind size specification for ethanol fermentation, the milled cob was further ground for two 30 second vibration cycles using a Kleco machine (Garcia Manufacturing, Visalia, Calif., Model no. KLECO 8200).

### Dry Ground Sample Preparation

Samples were dried before analysis by lyophilization for at least 8 hours. Samples were milled to pass through a 0.5 mm screen. Dried samples were cut into manageable pieces and ground on a Perten® 3600 Disc Mill (setting 1; Perten Instruments AB; Huddinge, Sweden). The grinding procedure was slightly varied depending on the sample to grind samples to a reasonably fine consistency prior to analysis.

Extraction of Sugar with Water

Finely powdered cob samples were accurately weighed out (100-130 mg) in 15 mL tubes. Duplicate experiments were prepared with each sample. To each tube ddH₂O was added to a final concentration of 10 mg of cob sample/1 ml of water. The samples were incubated for 1 hour at room temperature with gentle rotation. Then the tubes were centrifuged for 15 min at 4,000 rpm. Next, 0.8 ml of clear supernatant was transferred to a spin filter while the pellet was kept for total starch analysis. The filtered supernatant was centrifuged at 5,000 rpm for 15 min and then stored at −20° C. until sugar analysis by HPAEC.

### Sugar Analysis by HPAEC

The filtered samples stored at −20° C. were thawed and diluted 20 fold (10 μL of sample+190 μL of ddH₂O) in the 200 μL HPLC vials or plate. Standard sugar [Glucose+Fructose+Sucrose] mixture at different concentrations [0(H2O), 0.01, 0.02, 0.04, 0.10 mg/mL] was used as standards for expected sugar components. HPAEC was run with the following program:

Column: Carbopac PA200 Carbohydrate column

Flow rate: 0.4 ml/mL

Sample volume injected: 10 μL

Reagent: A: Water; B: 1 M NaOH; C: 1M NaAc

Gradient:
0 min: 100 mM NaOH
30 min: 100 mM NaOH+80 mM NaAc
32.5 min: 100 mM NaOH+900 mM NaAc
35 min: 100 mM NaOH
45 min: 100 mM NaOH
   The % of each sugar and sum of the three sugars in the dry samples was calculated by comparison of the integrated peaks to that of the standards.
   Removal of Soluble Sugars from the Pellet for Starch Analysis

To each sample pellet 10 mL of 80% ethanol was added and vortexed to completely suspend the pellet. The suspended pellet was then incubated in water bath at 80-85° C. for 5 min. Next the suspended pellet was centrifuged at 3,000 rpm for 10 min at room temperature and the supernatant was discarded. An additional ethanol wash step was repeated once more to completely remove any soluble sugar residues.

### Starch Digestion of Plant Tissue

3 mL of a 1:30 dilution of thermostable α-amylase (MEGAZYME, Wicklow, Ireland) in 50 mM MOPS Buffer (pH 7.0) was added to the sample pellet. The pellet in each tube was completely suspended by vortexing or pipeting up and down several times. Amylase catalyzed digestion was carried out in a 100° C. water-bath for 12 min, vortexing every 4 minutes. The reactions tubes were transferred to a 50° C. water-bath. 4 mL of 200 mM NaOAc buffer pH=4.5 was add to each sample, followed by addition of 0.1 mL amyloglucosidase (Megazyme). Reaction tubes were periodically inverted to mix contents and allowed to incubate in a 50° C. water bath for 30 min. The sample volume was adjusted to 10 mL with water in a 15 mL tube followed by centrifugation at 3,000 rpm for 10 min at room temperature. This supernatant contains the solubilized sugars that were digested out of the sample.

### Glucose Assay of Starch Digests

20 μL of starch digest supernatant was pipetted to a 96 well assay plate well in triplicate per replicate. Glucose standards [0 (Water, =blank), 0.1, 0.2, 0.3, 0.4, 0.5 mg/mL] were also added to the same 96-well assay plate. Glucose Oxidase reagent (200 IA) was added to each well and incubation was carried out at 37° C. for 20 min. Absorbance at 500 nm was read. Samples with OD greater than 1.0 were diluted 2× fold [10 μL of starch digest supernatants+10 μL of water] and the glucose assay was repeated with standards. The glucose concentrations in the samples were calculated based on the standard curve. The following calculations may be used:

The total amount of glucose (mg)=glucose concentration (mg/mL)×10 mL.

The total amount of starch (mg)=total amount of glucose (mg)×164/182.

The total amount of starch (% dry weight)=total amount of starch (mg)/sample weight (mg)×100%.

### Results

Cob tissue from T1 events comprising RNAi cassettes from constructs 17305, 18222, 18278, 18286 and 18288 showed an average increase in starch content (%/mg of dry weight) compared to the average starch content of null cobs (see Tables 10-15). Null cobs were observed to have an average starch content of 0.89% starch per mg of dry weight (Table 10). In comparison, events from construct 18286 (TrpA-R1) surprisingly showed a 2.8× increase of starch over null cob tissue with 18286 events having a average starch content measuring approximately 2.51% per mg of dry weight (Table 14). T1 events from construct 18288 (Zm015970-R1) showed the second highest increase in starch at approximately a 2.1× increase of starch in cob over null cob tissues with 18288 events having an average cob starch content measuring approximately 1.90% per mg of dry weight (Table 15). Cob tissue generated from construct 17305 (OsMADS13-R1) contained 1.79% starch per mg of dry sample, a 2× increase over null cob events (Table 11). T1 events generated using construct 18278 (OsMADS14-PWD) showed an average starch content of 1.59% per mg of dry sample, a 1.79× increase in starch over null cobs (Table 13). T1 events generated using construct 18222 (OsMADS13-AMY3) showed an average starch content of 1.27% per mg of dry samples, a 1.43× increase in starch over null cobs (Table 12). Surprisingly, all constructs downregulating a maize endogenous starch degradation enzyme showed an increased accumulation of starch when compared to null cobs. Noticeably, constructs suited for the downregulation of R1 in cob tissue resulted in the highest amount starch accumulation in cob. There was no significant difference (P=0.33) in total sugar content or in the amount of individual sugars (glucose, fructose, and sucrose) observed in cob samples where R1 had been down regulated compared to null controls (see Table 11).

TABLE 10
Total starch estimation in cob samples from T1 null events

| | | Starch Content [%/mg dry weight] | | |
|---|---|---|---|---|
| | | Rep 1 | Rep 2 | average |
| MZBF080322A035A-15 | zero | 1.17% | 1.21% | 1.19% |
| MZBF080322A035A-24 | zero | 0.70% | 0.64% | 0.67% |
| MZBF080322A072A-10 | zero | 0.81% | 0.75% | 0.78% |
| MZBF080322A100A-04 | zero | 0.99% | 0.86% | 0.93% |
| MZBF080322A100A-09 | zero | 0.88% | 0.91% | 0.90% |

TABLE 11
Total starch estimation in cob samples from
T1 events with 17305 construct

| | | Starch Content | | |
|---|---|---|---|---|
| | | [%/mg dry weight] | | |
| | | Rep 1 | Rep 2 | average |
| MZBF080322A035A-16 | heterozygous | 1.46% | 1.36% | 1.41% |
| MZBF080322A072A-11 | heterozygous | 2.04% | 1.99% | 2.01% |
| MZBF080322A100A-06 | heterozygous | 2.09% | 2.05% | 2.07% |
| MZBF080322A072A-01 | homozygous | 1.93% | 1.86% | 1.90% |
| MZBF080322A072A-21 | homozygous | 1.86% | 1.80% | 1.83% |
| MZBF080322A072A-23 | homozygous | 1.86% | 1.76% | 1.81% |
| MZBF080322A100A-20 | homozygous | 1.47% | 1.54% | 1.50% |

TABLE 12
Total starch estimation in cob samples from
T1 events with 18222 construct

| | | Starch Content [%/mg dry weight] | | |
|---|---|---|---|---|
| | | Rep 1 | Rep 2 | average |
| MZBF093606A040A | heterozygous | 1.11% | 1.05% | 1.08% |
| MZBF093606A022A | heterozygous | 1.64% | 1.46% | 1.55% |
| MZBF093606A054A | homozygous | 1.23% | 1.12% | 1.18% |

TABLE 13
Total starch estimation in cob samples from
T1 events with 18278 construct

| | | Starch Content [%/mg dry weight] | | |
|---|---|---|---|---|
| | | Rep 1 | Rep 2 | average |
| MZBF093523A002A | heterozygous | 2.00% | 2.03% | 2.01% |
| MZBF093523A032A | heterozygous | 1.32% | 1.38% | 1.35% |
| MZBF093523A048A | homozygous | 2.54% | 2.36% | 2.45% |
| MZBF093523B049A | homozygous | 1.60% | 1.58% | 1.59% |
| MZBF093523A045A | homozygous | 1.11% | 1.06% | 1.09% |
| MZBF093523A034A | homozygous | 1.15% | 1.25% | 1.20% |
| MZBF093523A018A | homozygous | 1.43% | 1.55% | 1.49% |
| MZBF093523B004A | homozygous | 1.53% | 1.58% | 1.56% |

TABLE 14
Total starch estimation in cob samples from
T1 events with 18286 construct

| | | Starch Content [%/mg dry weight] | | |
|---|---|---|---|---|
| | | Rep 1 | Rep 2 | average |
| MZBF093527A003A | heterozygous | 2.39% | 2.64% | 2.51% |
| MZBF093527A005A | heterozygous | 3.28% | 3.31% | 3.29% |
| MZBF093527C035A | heterozygous | 1.78% | 1.69% | 1.73% |

TABLE 15
Total starch estimation in cob samples from
T1 events with 18288 construct

| | | Starch Content [%/mg dry weight] | | |
|---|---|---|---|---|
| | | Rep 1 | Rep 2 | average |
| MZBF093516B006A | heterozygous | 1.67% | 1.40% | 1.53% |
| MZBF093516B010A | heterozygous | 1.85% | 2.29% | 2.07% |
| MZBF093516A005A | heterozygous | 2.22% | 1.99% | 2.10% |

TABLE 16
Sugar estimation in cob samples from
T1 events with 17305 construct

| | | | | | Total |
|---|---|---|---|---|---|
| | | Glucose | Fructose | Sucrose | Sugar |
| MZBF080322A035A- | hetero- | 4.37% | 4.45% | 0.63% | 8.88% |
| 04 | zygous | | | | |
| MZBF080322A035A- | hetero- | 3.71% | 4.90% | 2.21% | 11.07% |
| 16 | zygous | | | | |
| MZBF080322A072A- | hetero- | 3.46% | 4.83% | 2.39% | 10.50% |
| 11 | zygous | | | | |
| MZBF080322A100A- | hetero- | 5.87% | 8.75% | 2.94% | 17.73% |
| 06 | zygous | | | | |
| MZBF080322A072A- | homo- | 5.54% | 7.75% | 1.58% | 14.93% |
| 01 | zygous | | | | |
| MZBF080322A072A- | homo- | 6.33% | 7.98% | 1.32% | 14.86% |
| 21 | zygous | | | | |
| MZBF080322A100A- | homo- | 2.99% | 3.78% | 1.35% | 7.76% |
| 20 | zygous | | | | |
| MZBF080322A035A- | zero | 2.04% | 4.90% | 1.60% | 8.30% |
| 15 | | | | | |
| MZBF080322A035A- | zero | 2.94% | 3.17% | 0.51% | 6.20% |
| 24 | | | | | |
| MZBF080322A072A- | zero | 6.61% | 7.62% | 3.27% | 17.52% |
| 10 | | | | | |
| MZBF080322A072A- | zero | 6.11% | 7.87% | 3.74% | 18.37% |
| 31 | | | | | |
| MZBF080322A100A- | zero | 4.66% | 5.85% | 3.21% | 13.72% |
| 04 | | | | | |
| MZBF080322A100A- | zero | 2.56% | 3.21% | 1.11% | 6.61% |
| 09 | | | | | |

### Example 4

### Fermentation of Maize Cob Tissue Samples

The following ethanol fermentation method includes a high temperature saccharification step with the addition of Thermostable α-amylase: (3,000 U/mL at pH=6.5 and 40° C.) (bottle 1 in kit) Diluted lmL of the enzyme to 3 mL using the 50 mM MOPS buffer) and amyloglucosidase (3,300 U/mL at pH=4.5 and 40° C.) (bottle 2 in kit) used as provided with Megazyme's Total Starch Kit (MEGAZYME, Wicklow, Ireland). Fermentations were carried out at 20% solids for 17 hours.

### Cob Sample Preparation:

Cobs were prepared as described in the previous examples. Fresh cobs were frozen at 80° C., photographed, and the seeds were removed using a paint scrapper. Each cob was then sliced into slices less than ¼ inch thick. Slices for each cob were placed in a weigh boat and dried using the lyophilizer. Two of the slices (one from the middle and one from the end) were put aside for Lugol's Iodine staining. The remaining cob slices were ground using a centrifuge mill. To achieve the required grind size specification for ethanol fermentation, the milled cob was further ground for two 30 second vibration cycles using a Kleco machine (Garcia Manufacturing, Visalia, Calif., Model no. KLECO 8200).

### Yeast Preparation

Ethanol Red Yeast from Fermentis (FERMENTIS Division of S.I.Lesaffre, France) was prepared for fermentation prior to each experimental setup. 1 g of solid yeast; 5 g of 1% glucose solution, was weighed into a 50 ml conical tube. The tube was lightly tapped on a vortex until all of the yeast was suspended. The mixture was then incubated at 30° C. for 30 minutes with continuous shaking at 120 rpm. The yeast was lightly vortexed, removed and diluted to 5× with DI water.

### Dry Ground Sample Preparation

Samples were milled to pass through a 0.5 mm screen. Dried samples were cut into manageable pieces and ground on a Perten® 3600 Disc Mill (setting 1; Perten Instruments AB; Huddinge, Sweden). The grinding procedure was slightly varied depending on the sample to grind samples to a reasonably fine consistency prior to fermentation.

### Fermentation of Maize Plant Tissue Samples

Two 15 ml disposable centrifuge tubes were labeled for each individual sample, including the control (null). 750 mg of ground sample was weighed into each tube and the weights were recorded for calculations. A single 2 mm metal ball was added to each tube and each tube was re-capped until the fermentation was started. Each sample was washed three times by adding 5 ml of 50 mM MOPS buffer, vortexing for 30 seconds, and then centrifuging at 3000 rpm for 5 minutes. 3 ml of supernatant from the top was discarded each time and another 3 ml of fresh buffer was added between each wash. After the third wash, 3 ml of the supernatant was removed and the tubes were set aside. One tube was set up for a media only (no enzyme, no yeast) check sample. Fermentations were carried out at 20% solids by adding water to each tube. The final volume for each fermentation was approximately 3 ml. Each tube was vortexed to re-suspend the solids and sealed with Paraflim®. A small hole in the top of each tube was poked using a 16.5 gauge needle. Fermentation was initiated by rotating the tubes at speed setting of 4 in tissue culture rotor (CEL-GRO, LAB-LINE) in the 30° C. incubation room. Fermentation reactions were carried out as depicted in Table 12 below.

TABLE 12
Preparation of Fermentation Reactions

| | | | |
|---|---|---|---|
| Solids | Flour | 750 | (mg) |
| Addition 1 | Diluted thermostable α-amylase | 519.25 | μL |

Heated at 100° C. for 12 minutes in a water bath.
Vortexed every 4 minutes.

| | | | |
|---|---|---|---|
| Addition 2 | Amyloglucosidase | 200 | μL |

Heated at 50° C. for 30 minutes in a water bath.
Vortexed every 10 minutes.

| | | | |
|---|---|---|---|
| Addition 3 | YP, 10X (YE 100 g/L, Peptone | 300 | μL |
| | 200 g/L) | | |
| | Tetracycline (10 mg/ml) | 0.75 | μL |

Tubes were vortexed and spun down at 3000 rpm for 5 minutes. 100 μl
of supernatant was removed and placed in a 0.4 μm filter spin column
for zero time point data (T0). The T0-hour aliquots were spun down in
the spin column at 6000 rpm for 10 minutes. T0 hour samples were
placed in a 4° C. refrigerator. The remaining tubes were resuspended and
carried on to Addition 4 by adding yeast.

| | | | |
|---|---|---|---|
| Addition 4 | Yeast | 20 | μL |

Fermentations were allowed to proceed for 17 hours at 30° C.

Fermentations were sampled from each reaction tube after 17 hours of incubation by centrifuging tubes for 5 minutes at 3000 rpm and removing 100 μL of sample.

### HPLC Analysis of Ethanol Yield

Ethanol, in sample from fermentation broth, was separated through a Micro-Guard Cation-H Refill Cartridges 30×4.6 mm (Bio-Rad, Cat no. 125-0119) and Aminex HPX-87H Ion Exclusion Column 300×7.8 mm (Bio-Rad, Cat no. 125-0140) and detected with RI detector using High Performance Liquid Chromatography (HPLC, Waters Alliance).

The filtered T0 samples and 17 hour samples were transferred to the proper HPLC vials. 0.12 μL of 25 mM H₂SO₄ was added to 88 μL of sample in each vial to remove the negative peak from the HPLC analysis. Five HPLC standards (0%, 0.25%, 0.5%, 0.75% and 1% ethanol) were analyzed to create a standard curve, which was used to determine the EtOH content in the sample.

### Results

Ethanol yields from starch fermentation with T1 cob samples where maize R1 has been downregulated was observed to be approximately two fold or higher than observed yields using null cob samples (See Table 13)

TABLE 13
Net Ethanol Yield at 17 hours of Fermentation

| | | Ethanol | Ethanol |
|---|---|---|---|
| | | (% v/v) | (average % v/v) |
| Null control | MZYE013 | 0.18 | 0.17 |
| | | 0.16 | |
| 17305 event I | MZBF080322A100A-6 | 0.45 | 0.43 |
| | | 0.41 | |
| 17305 event | MZBF080322A072A- | 0.36 | 0.34 |
| II | 11 | 0.330 | |

The amount of starch (fermented starch, % per g dry weight of the cob sample) fermented to produce ethanol was calculated from the ethanol yield shown in Table 1.

Starch content (estimated starch content, % per gram dry weight of cob sample) was estimated using Megazymes's Total Starch Assay Kit (see Table 14).

TABLE 14
Estimated Fermented Starch in T1 Cob Samples

| | | Fermented | Fermented | Estimated Starch |
|---|---|---|---|---|
| | | Starch* | Starch* | Content # |
| | | (%/g) | (%/g, average) | (%/g) |
| Null control | MZYE013 | 1.00 | 0.94 | NA |
| | | 0.89 | | |
| 17305 event I | MZBF080322A100A-6 | 2.49 | 2.38 | 2.07 |
| | | 2.28 | | |
| 17305 event II | MZBF080322A072A-11 | 2.00 | 1.90 | 2.01 |
| | | 1.81 | | |

1. A method for increasing starch content in cob tissues of a maize plant comprising:
a) inserting an expression cassette into a maize plant cell comprising a polynucleotide operably linked to a regulatory element that ensures transcription in maize cob tissue wherein expression of the polynucleotide decreases activity of one or more endogenous starch degradation enzymes in maize cob tissues;
b) regenerating transgenic maize plants from the maize plant cell of a); and
c) producing said cob tissue with increased starch content.

2. The method of claim 1, wherein said endogenous starch degradation enzyme is selected from the group consisting of alpha-amylase, glucan water dikinase, phosphoglucan water dikinase (PWD), limit dextrinase, isoamylase, beta-amylase, chloroplastic glucan phosphorylase, disproportionating enzyme, chloroplastic maltose transporter protein (Mex1), chloroplastic glucose transporter protein and chloroplastic triose phosphate transporter protein.

3. The method of claim 1, wherein said endogenous starch degradation enzyme is selected from the group consisting of alpha amylase, glucan water dikinase, phosphoglucan water dikinase (PWD), Chloroplastic alpha amylase (AMY3) and beta-amylase.

4. (canceled)

5. The method of claim 1, wherein said endogenous starch degradation enzyme is an alpha-amylase.

6. The method of claim 1, wherein said polynucleotide that decreases activity of one or more starch degradation enzymes is a RNAi.

7. The method of claim 1, wherein said regulatory element is a cob tissue preferred promoter.

8. The method of claim 1, wherein said regulatory element is a OsMADS promoter.

9. The method of claim 1, wherein said regulatory element is any one of the promoters from the group consisting of a OsMADS13 promoter, a OsMADS14 promoter, a TrpA promoter, and a Zm015970 promoter.

10. (canceled)

11. A method of improving the yield of free sugars from plant biomass for a biomass conversion method, said method comprising:
a) inserting an expression cassette into a maize plant cell comprising a polynucleotide operably linked to a regulatory element that ensures transcription in a maize cob tissue wherein expression of the polynucleotide decreases activity of one or more endogenous starch degradation enzymes in maize cob tissue.
b) regenerating transgenic maize plants from the maize plant cell of a); and
c) growing said transgenic maize plants under conditions in which the polynucleotide is expressed, wherein expression of the polynucleotide results in an increase in starch content in cob tissues of said plant and,
d) using said transgenic maize plant in a biomass conversion method.

12. The method of claim 11, wherein said endogenous starch degradation enzyme is selected from the group consisting of alpha-amylase, glucan water dikinase, phosphoglucan water dikinase, limit dextrinase, isoamylase, beta-amylase, chloroplastic glucan phosphorylase, disproportionating enzyme, chloroplastic maltose transporter protein (Mex1), chloroplastic glucose transporter protein and chloroplastic triose phosphate transporter protein.

13. The method of claim 11, wherein said endogenous starch degradation enzyme is selected from the group consisting of alpha amylase, glucan water dikinase, PWD, AMY3 and beta-amylase.

14. The method of claim 11, wherein said endogenous starch degradation enzyme is a glucan water dikinase.

15. The method of claim 11, wherein said endogenous starch degradation enzyme is a alpha-amylase.

16. The method of claim 11, wherein said polynucleotide is operably linked to a cob tissue preferred promoter.

17. The method of claim 16, wherein said polynucleotide is operably linked to a OsMADS promoter.

18. The method of claim 17, wherein said polynucleotide is operably linked to any one of the promoters from the group consisting of a OsMADS13 promoter, a OsMADS14 promoter, a TrpA promoter or a Zm015970 promoter.

19. The method of claim 11, wherein said plant cell further comprises a second polynucleotide operably linked to a regulatory element that ensures transcription in plant cells wherein expression of the second polynucleotide encodes a processing enzyme and said processing enzyme is expressed so that it does not come in contact with its' substrate.

20. The method of claim 11, wherein said plant further comprises a polynucleotide operably linked to a regulatory element that ensures transcription in plant cells wherein expression of the polynucleotide decreases activity of one or more endogenous starch degradation enzymes in plant green tissue wherein said green tissue has increased starch content.

21. The method of claim 11, wherein said biomass conversion method is a non-animal feed biomass conversion method.

22. The method of claim 20, wherein said non-animal feed biomass conversion method converts carbohydrates to one or more biofuels.

23.-43. (canceled)


SEQUENCE LISTING (20 sequences)

SEQ ID NO: 1 - DNA, 500 bp, Zea mays
  misc_feature (1)..(500): maize alpha amylase RNAi fragment
agggcgtgctgcaggcggccgtccagggcgagctgtggcgcatgaaggacggcaacggcaaggcgcccgggatgatcggctggctgccggagaaggccgtcacgttcgtcgacaaccacgacaccggctccacgcagaactcgtggccattcccctccgacaaggtcatgcagggctacgcctatatcctcacgcacccaggaactccatgcatcttctacgaccacgttttcgactggaacctgaagcaggagatcagcgcgctgtctgcggtgaggtcaagaaacgggattcacccggggagcgagctgaacatcctcgccgccgacggggatctctacgtcgccaagattgacgacaaggtcatcgtgaagatcgggtcacggtacgacgtcgggaacctgatcccctcagacttccacgccgttgcccctggcaacaactactgcgtttgggagaagcacggtctgagagttccagcggggcggcaccactccgag

SEQ ID NO: 2 - DNA, 500 bp, Zea mays
  misc_feature (1)..(500): maize beta amylase RNAi fragment
gctacagaaccatagcacacatgctcacaaggcatcgtgctagcatgaacttcacttgtgctgagatgagggacagtgaacagagttcagaggcgaaaagtgcacctgaggaactggttcaacaggtgctgagtgctggatggagagagggcctaaatttggcatgtgaaaatgcactcaaccgatatgatgcaacagcttacaacaccatcctcaggaatgcaagacctcaaggcatcaacaagaatggccctccagaacacaagttgcacggattcacctacctccgagtatctgatgaactgttccaggaacagaactacaccactttcaaaacttttgtcaggagaatgcatgctaacctggattataatccaaatgtcgatccagttgcaccattggaaagatcaaaggcagagataccaattgaagaaatcctagaagtagcacagccaaaattggagccatttcccttcgacaaggacaccgacctaccagtt

SEQ ID NO: 3 - DNA, 330 bp, Zea mays
  misc_feature (1)..(330): maize R1 RNAi fragment
gatgaccttgactctcccaagttacttggttacccaagcaagccaattggtctcttcataaggcaatcaatcatcttccgttccgactccaacggtgaggacctggaaggttatgctggagcaggattatatgatagcgtaccgatggatgaggaggatgaggttgtacttgattacacaactgaccctcttatagtagaccgtggattccgaagctcaatcctctcaagcatagcacgggctggccatgccatcgaggagctatacggttctcctcaggacgtcgagggagtagtgaaggatggaaaaatctatgtagtccagacaaga

SEQ ID NO: 4 - DNA, 320 bp, Zea mays
  misc_feature (1)..(320): maize PWD RNAi fragment
gatctactgctaatgtggaggatttggctggaatgtctgctgctggactctatgaatcaattcctaatgtcagtctctctgaccctagatcctttggagctgcggttggacaggtttgggcatcattatacacaaggagagcaatccttagccgtcgagcagccggtgtacctcagagagacgcaaagatggcggttctcgtgcaggaaatgctgcagccagatctctcttttgtgcttcatacaattagcccagttgaccatgatcccaaattagtagaggctgaggttgcccctggtcttggagaaaccctcgcttct

SEQ ID NO: 5 - DNA, 320 bp, Zea mays
  misc_feature (1)..(320): maize AMY3 RNAi fragment
ggaatgatcttaaagaatggctttgctggatgagaaaggaagtcggctacgatggatggagacttgactttgttcgtggtttctggggtggatatgtcaaggactatttggaagcaagtgaaccatactttgcagtaggagagtactgggactccctcagttatacttatggtgaaatggactacaatcaggatgcccacaggcagagaattgttgattggataaatgctacaaatggaactgctggcgcatttgatgttaccactaaaggaatacttcatgcggcgcttgaaagatcagagtattggcgcctgtccgat

SEQ ID NO: 6 - DNA, 1894 bp, Oryza sativa
  promoter (1)..(1894): OsMADS13 cob preferred promoter
ttccaaaattaagcacacacatttgcaagaactagctaggcatgcatatatgataattaaccggcaagttgacttcagttattctgcagatgtactaaacacataacaagggatgatcagttgcttatttttttcataacttgctaggttgcttataactccagccttctggacatcgaccaatctctaaacatactttagcagtgcctacaaagtacaaacaactaaatacctctctgcagatcagtgtttctaggcacaaattacacaagatagaaaaaaggagaggttataaattcttgcttaaagaatatacatgtaaagatgtctaaatagctataaatgggtaagcaagatagcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgctaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctctggtctccttttttgttgatcagttgagagcaagaag

SEQ ID NO: 7 - DNA, 2195 bp, Zea mays
  promoter (1)..(2195): TrpA cob preferred promoter
ccaaaagtcttgaaaaaattcagcggggaggccattagggcaggggtactgttatgttttaaagagaacaccactttcttgatctcttctaaagagaaatgttttgtaagaaagatcctgtcctcctcatccaaccttttcatcggcaaatttttcatagagatattagaggcaagagaggggccaaaaagatccatgtaaatggaagtggccacctggttgatacctccctcatcttcaacagaaaatccattatgaaaaagtgaatggattttaaactcttctttttcttcccttttgcaatgagctgaaaatatctggtattattctcatcaccctcattaatgaatctgtccctagcaatttgctttctcttgatcccttctgcagccaccatgtttcttaaattccactccatatcaagcttttccaatctatcagaatctgagatggctgcaatctctctcattttctcaaggatatcgatgttatccataaggtatttcttgaacttcttatatttcccttcgacatttatattccatcctttcaacatttttttgttcaatcttttttgtttttttcctttccaaacatcgatacatttcctgctcctcacaggtaaggacgagctttcaaaaaaccttctgctttaaagtcaggtctgagcctccagcaaagctcacatatctaaagtccctcttcttagttgggacagagtcagtgctaagacacatgggaacatgaccagaaaaaaaaaatcatatttagcccagagacaacaatattcttgtactgcaagtctcgttatgggctagcaaaggaatctacccaacttctcaaatgtgttgggatgtcaagtatatagactattcatcagttccaactctatcaaactgtgcagctcaattatagagttgaataaagtgctccatctatttgttcttatcctcatatttggttaagatattaaaatcacctcccaccaacatttaaagtgcaccatttaaagtggctcgcgagcaccaaaccgctgaaaaccggaaatgtttagcacgttggcagcgggccccttttctatctcatcgtgttcttcgttgtccaccacggcccacgggccaacgctcctccatcctgtagtgtagagtatattccatttgcgaccgagccgagcatcgatccagccacactggccactgccagccagccatgtggcactcctacgtatactacgtgaggtgagattcactcacatgggatgggaccgagatattttactgctgtggttgtgtgagagataataaagcatttatgacgattgctgaacagcacacaccatgcgtccagatagagaaagctttctctctttattcgcatgcatgtttcattatcttttatcatatatatataacacatattaaatgattcttcgttccaatttataattcatttgacttttttatccaccgatgctcgttttattaaaaaaaatattataattattgttactttttgttgtaatattgtttagcatataataaactttgatactagtatgtttccgagcaaaaaaaaatattaatatttagattacgagcccattaattaattatattcgagacaagcgaagcaaagcaaagcaagctaatgttgcccctgctgtgcatgcagaggcccgctcttgctataaacgaggcagctagacgcgactcgactcatcagcctcatcaacctcgacgaaggaggaacgaacggacaggttgttgcacagaagcgacagatctgctttcgcgcccaaaacgtcctcctcctcctcgctgtcctcggcgttgcaggcagctcagtcgccgccgctgctcctgaggcggatctcgtcgaccgcaacaccgagacggaggtacgacgcggccgtcgtcgtcactaccaccaccactgctagagctgcggcggctgctgtcacggttcccgccgccccgccgcaggcgggccgccgccgccggtgccaccaaagcaagcggcggcacccgcagaggaggagccgtccggtgtcggacaccatggcggcgctcatggccaagggcaaggttcgtatagtacgcgcgcgtgtcgtcgtcgttattttgcgcataggcgcggacatacacgtgctttagctagctaacagctagatcatcggtgcagacggcgttcatccc

SEQ ID NO: 8 - DNA, 2670 bp, Zea mays
  promoter (1)..(2670): Zm015970 cob preferred promoter
ggcgctcccagccgtgaaaacgcgtatcgcgccaccaccggggacccctcccgcgccgggacgagagagctgatcgacggtcgctagaggagagaggaggtcgacagacgacaggggacggcgcgcgtcaggtggtggggctgaccgctgacggggacacgcgggggtgcgcgcgtcgtccgttcgatcgatctgtcgtcggcccggccgttcggcgagatagccaggccagagcttgccttgccgccggcggtgaaacggcgacagcttggctagagaacacgaggcaccgcgttttgatcgcgcctctcgtccccgggcaatcgctgttgacgagtatggccggcaggccgcggcacgcgagggccgtccagtttccggaggtaaggagggagcttcctactcttgtaggcatcgtccttttcctttgaaaatctgttgtatacgtttccttctttttcccttcaaacgactggtttatttacacatacatttcaggctccgcattttagacatcaaggaaaagtatcttttggagtatctccaacaatgccttaaactaaaatatgatactaaaaactactccaacagtgcatcattttataaaatttggttaaaattttatagggcgttctctcaagtaactcaaatatactacaccgcagtgatctgtcctataatctagatgtgaggttttaatattgaaactgaatgttttgttggtgacctaaattctataaaatatactcattttaaattatagagcacttttataggtcattttgttagagatgatcttatacattgtgtttaaccgcagcaatcggctgtggatttatttgtttttagatttgggcaatatgtttgtatagacttcgttttattgttgaattattcaaagctagctagagcaactgatcatatactcgtatgagtctaacacatgttcatgccctacaaaggaagaaagtctaacacatgttcatgttcagaattgagagaaaaaaagaaacttttagctacagtacaacttacaagtcttgcaggaacacaggatgcagcctacattacatatacgtctctcttggtcacaagacggcatcctgtgatgagaatatttccaggaagattcttttgtcaatttctagccttgcagaaattctcggtaagctattttcagacttgtcacaatttaagtcgacatcacaaacaaagcaaaaaaaaacgcagtccctcattgttgtgatttgtgaataagaaaatcgtgtggagaaatttcaagggaatgtgttgtggcacaaataaactttgtagatgtacaaaaatgcaaaggggattagtacaggtaggactactcaattaaataacatttgaaattaaattttttcactaaaataaatattcattgttccacggtcttattgtggacactaaagtgccgtttgattcacaaaatgtaacgtaaatgataacggtaatgctttacactcgatatcagcggtaacaagtttgaataagacaatatcagttttgtaaacggttggatttaacaaacatggttgtgtgcatgcatgcgagagggggagacgaggggcgaaacgaggatccaaaattagggggttaaattagattaaggggctgttagaagtgttagaagcatttttacattatttacatgagaattagcttaaaaactaacatgtcctatagaattaagcattaaaaccgcatgagtaataaatatataatacaaatacaaaaaaaaaactgagaagaaatggcacagatccgaagtggcaaattgtcttccttcgccttcgcgcgctcttctataaatacctcccctctcaccattcccttgccatcagacaagagacacacacacaccacacgcagtcacgcacagcctcggcctccgcagctgcaggcgcctgctgctcttcgacaagaccccaagaactcgccaacctaacctaggtcagccacccttccttctaccttctgcgacaatgcaggccgccgccgcctccgtcaagtcctccaccaccgcggccgcggccgcggccacctgcaaggcgggcttcaagcccctgcacctccctcccctccccgccgccggctccaggccgctgtccctctccgtctccgcgcgtccgctgtaccgccaagaccatgtcctcgcgacaaccgtggcgatggcggcggcgggccgcagcgaccgcgccgcgtcccccgcgcctccgtccgccacagcggacggcgcccggccggtggtggaggtcgccgcgccggcggagaccgcgcgccgcgccaagatcggggtgtacttcgccacgtggtgggcgctgaacgtgatcttcaacatctacaacaagaaggtgctgaacgcgttcccgtacccgtggctcacgtcgacgctgtccctcgccgccggctccgccatcatgctcgcgtcctgggccaccaggatcgccgaggcgccccagacggacctcgacttctggaaggcgctcactcccgtgagtatcgatcggcgccgcttaattccttcctaaagatctctagcttcttcttctagatgcggctcgatctgtgtggtgctggattgctgatgcgagtcgtctcgtggtcgtggccggccggccgagcaggttgcgatcgcgcacacga

SEQ ID NO: 9 - DNA, 4856 bp, Oryza sativa
  promoter (1)..(4856): OsMADS14 cob preferred promoter
tggaagtttgaagaaattgaaacgatgtgatggaaaagttgaaagtttgaagaaaaaagttggaatctaaatagggcccaacatactcaaggctttggaatattactcacacacacacacacacaaattcatgtgggtcaaatagcttgtcctttagaattttggaatttcccccattctccaactagaatatccaatgaaattattgcgtttaaaagaaagagttgggcggtgacgatatggggttttatcaactttcaaaatgaacacctatgaaatttctacttaacctcaacttcaatatgagaaatgtaaagtaaaaggaaaaattttctacaaaacatctaaaaaacatgatctttgctggtggacaccgttaaaaatgtagcttttgtcttagggcactaaaaaacgtgataatttgctggagaacatcacggcctttaaaatgtaaaatccacttaattgagaagagatgaggcatatgaaatgtttattttgccctcctcttcttcctcgccactcatgtcatcgcctcgctgcctgctaataggggtttgagtgagagagcacaaagattgagaggcggggatatgatcagttcgaactgagacatactccttcgttcacatatgcgggataggggcctcctaaaatatttttctatcaaataggatgaaaacaataaaataatcgatgtttatgttcttcagcaaattaccatatttttatagtgttctcggaaaatccacgttttgatttagatatactgtagcaaatttgccaaattaaaactaaccggtttaattaattttaggttttagcaagtactgtacgctcttcaagttgtggcaactactttgattgcagctacagtacaccccccgatcgagagcgagaccgagggttacgtggtgggcccagcgcggcagccacactgtaggagtgacagtgctaggctacagctactgtgctgggcgtagtagttgttccgctcactgtgcagatgaggtcatgcgctcatcgatccccctcctctcctcctcctcctctctgtcctctgccatcccccccacccgtcacgtgaggcgacgccgccgacctgatggctcgacatgccccccgtcccggtcgacgcgacgtgcactgtgaccggccggaagaacgccccccctgccagccggcgacacgcacgcgtccggccaaggctgcctagctagcgtatcgctcccccagctaccggcctcgccgtgtggcccggcgccccggcgtgcgtacgcgtcgggtacgtaggaaacacggcggcagcgtcgaggcgaggacggggaagcggctggaccggcgggccgggggaagagaggcagccatcctgtactctcctacgtacagccaccttacctgttgcgtcaacgagagcttgggcgttgctcgctacagccgctggctagcttagcttatagctagtctccgcttagcctgtgggcataaaagcacccggcaccagcgtccgctcgacacgtcgcgattccgcctctgtgcgcgctgctgcccgggccgcaccgtgtgatacgtacacaacctcgggttcgacgcccccgccggcccggtcgcttccctcgcctcgctgttaacgtcgctgttacaagcttgcgaattgcgattgtgcgagcgctcgcggcggtattaatttttgctatacctggggcgatggaaacgtaacgtactactactacgtgacctagggtgagagacgagacgagacgagaatatgagatgagatccaaattcaatctgcctctctaccccacgaaagaaaagattcttctcttcgtcgacgacccggcctctcgctctcgttgcctttctttcgcgctcagtctctctcggccaattaagctgtagatcggccaccaccaccaccaccaccaccaccatccacagggcgctgcaagctgggccaggcctctctctatctctctctcttgagaggagagattaagtagggcatagagagaaaaaagctactcctaattaagccacagcttgagcagatcagctgcaggccggccggtgtgcgtgtgtgacagtaggagagagagctagcgagagagagcaaggaatcggaattgatttcatgcactgatcgatccatccatcatgcagatgctgcatggatgagaagagctagcagctaagctttgctgaagaaaattcttcagaggtttgtgttgatttgctggtctgctagagatcgatctagagagagagagaggaagggaagactgaagagagatctcgagcgggagatcgagatcgggcgaggcaaggtggtgcttcagcggatcgagaacaagatcagccggcaggtgacgttcgccaagcggcggaacggcctgctcaagaaggcctacgagctgtccatcctctgcgacgccgaggtcgccctcgtcctcttctcccacgccggccgcctctaccagttctcctcctcatccaagtaaacccactgcccctctcactcatctcagcttcctcctcttcccctaatcaaattctctcaacttgcttaacctgcactccccaaaccgcaatttgctccatctttgcgcgcacaatctgcaagaaatcacgaccgatctgaattttctcgcgcgtgtactgtatccgtcgtcgtccgtgcatgaacttatgggaggggccatgatttcgtacgtgttgttttttctacgcctgttcctttctttctgttctattgggcgaagctactgttcatgtaaaaaaggctacaaaagaacaaggcaaagaagacgaggttgtcctcggcaggagatccagacatgtacatatatgcgtatatataccgtgtgtgtatatatgtatgtgtgtcatagcaggcaagaaaaattaagaaaggggtttcatttgatgtaaatatcatggcgtgtacaaatgctagaatctgaattagcctaaaatttggaggatttaacttgatggagtttaaataatttacttggaagaatgccacttttagtactagctgttgatgagcaaaatttgctgaagaatgccgcttttgatcaaaatttgtcgtttacttatatattatcaaggatacttttgtgtgttaattataaacttgctgcaataaatatctcatgaacacaacacactagttaaactcaccaacatcgtcatgtgttaattcatctacggcttggaacttgcaagaactactttaacgtacaagctctgactttgtatatctgacagtaccaaaatactgttaccttatagctacgttggaaaagatcaactgtacaactactttcagctctataaaacaattcactgtaggcaaatgcaccgtccatatatatagacactctgtgaggtagaattgctaatttatagtatattattatattaacgtgtaattgacccatacaggataatcttcaaatttggggcagtttgcatgaaatgtggaactcacattagcaaaaattgcatattaaagaatacactgaccatgttaatatacatgctagtgagttatagtttgtatggggatgctaatatgttttggcctttttccccatatgaatgtatgctacattcttacaattaatatatacgttacaaagggaaatcaaatgacctagtagtttctattactgaattattgcacatttagtttttccaatcaatcactaaattgccataagggtccagtttttttgtctgtttcttgttggttctttgtcaagtatcatatatatatgtaccatatttttgtaggaatatgcattcgcgctagctaccgtatgatgatcacacaggtttcaacccttggttaacactgttaatttgttgttataaggggtatcatccatagagacagattactgtatttgaataagagatttctgtataataatgctatgccgttattggttctttatcaagtgccacagtaccttgtttttgtagtatatgtgttcactagctaccatgtgatgatcacacaaagtttcaccgttggtgaactgttaatttgtcatatgtgacatgcatcatccatagatacacgtttctcatattaataatttttaaatattgctaggaaaaaatggtgtcttattgggttataacacactagaatattgttttatttttatccttcatcctcagtgaaaaaggatgtacaattagtggcttggcacagactagggctgctttgttaattagatcaccttttatttttctcctaaaggtgaggggggggggggacgaaaacacttcggatggagcggcatacaaatttaaagctatattttccatatatttatttgcatttttcttgtttaatttggtataatcaattttctggtggcaccaaagcacctaagatggacttaggttgggttctttacttcttttagacactttctcaacttctattatctcgtttactttgtacgtttcccaaattgctaaacaatacgttttttataaaaatctataagaaagttgcttataaaaaaccatattaatctatttttaagtttttttaagctaatacttaattaatcgtgcgttactttgttttgtgtgcgagagggaacagttcccaaccctctccaatgaatgtaccctaaaggaggcttgattttttttttcctgaggaagaaaatgacaacgaaacattcacaaattgatcggaagacataattaacagctttattaatatactatttcttttaggggaaacatattttctgtatagtgtgctagttaaccatctatctctaattcatatttcagcatccttaagacgcttgagagataccacc

SEQ ID NO: 10 - DNA, 2345 bp, Artificial Sequence
  maize alpha amylase RNAi cassette
acaagtttgtacaaaaaagcaggctggatccagggcgtgctgcaggcggccgtccagggcgagctgtggcgcatgaaggacggcaacggcaaggcgcccgggatgatcggctggctgccggagaaggccgtcacgttcgtcgacaaccacgacaccggctccacgcagaactcgtggccattcccctccgacaaggtcatgcagggctacgcctatatcctcacgcacccaggaactccatgcatcttctacgaccacgttttcgactggaacctgaagcaggagatcagcgcgctgtctgcggtgaggtcaagaaacgggattcacccggggagcgagctgaacatcctcgccgccgacggggatctctacgtcgccaagattgacgacaaggtcatcgtgaagatcgggtcacggtacgacgtcgggaacctgatcccctcagacttccacgccgttgcccctggcaacaactactgcgtttgggagaagcacggtctgagagttccagcggggcggcaccactccgagctcgagacccagctttcttgtacaaagtggttggcgccaaagggcgaattcactagtaagcttgggcccgcggccgcaggtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttgttttttgaactgagaaaaatctttgggtttttggttggattctttcatggagtcgcgaccttccgtattcttctctttgatctccccgcttgcggattcataatattcggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgaggtatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcagggcgccaaccactttgtacaagaaagctgggtctcgagctcggagtggtgccgccccgctggaactctcagaccgtgcttctcccaaacgcagtagttgttgccaggggcaacggcgtggaagtctgaggggatcaggttcccgacgtcgtaccgtgacccgatcttcacgatgaccttgtcgtcaatcttggcgacgtagagatccccgtcggcggcgaggatgttcagctcgctccccgggtgaatcccgtttcttgacctcaccgcagacagcgcgctgatctcctgcttcaggttccagtcgaaaacgtggtcgtagaagatgcatggagttcctgggtgcgtgaggatataggcgtagccctgcatgaccttgtcggaggggaatggccacgagttctgcgtggagccggtgtcgtggttgtcgacgaacgtgacggccttctccggcagccagccgatcatcccgggcgccttgccgttgccgtccttcatgcgccacagctcgccctggacggccgcctgcagcacgccctggatccagcctgcttttttgtacaaacttgt

SEQ ID NO: 11 - DNA, 2345 bp, Artificial Sequence
  maize beta amylase RNAi cassette
acaagtttgtacaaaaaagcaggctggatccgctacagaaccatagcacacatgctcacaaggcatcgtgctagcatgaacttcacttgtgctgagatgagggacagtgaacagagttcagaggcgaaaagtgcacctgaggaactggttcaacaggtgctgagtgctggatggagagagggcctaaatttggcatgtgaaaatgcactcaaccgatatgatgcaacagcttacaacaccatcctcaggaatgcaagacctcaaggcatcaacaagaatggccctccagaacacaagttgcacggattcacctacctccgagtatctgatgaactgttccaggaacagaactacaccactttcaaaacttttgtcaggagaatgcatgctaacctggattataatccaaatgtcgatccagttgcaccattggaaagatcaaaggcagagataccaattgaagaaatcctagaagtagcacagccaaaattggagccatttcccttcgacaaggacaccgacctaccagttctcgagacccagctttcttgtacaaagtggttggcgccaaagggcgaattcactagtaagcttgggcccgcggccgcaggtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttgttttttgaactgagaaaaatctttgggtttttggttggattctttcatggagtcgcgaccttccgtattcttctctttgatctccccgcttgcggattcataatattcggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgaggtatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcagggcgccaaccactttgtacaagaaagctgggtctcgagaactggtaggtcggtgtccttgtcgaagggaaatggctccaattttggctgtgctacttctaggatttcttcaattggtatctctgcctttgatctttccaatggtgcaactggatcgacatttggattataatccaggttagcatgcattctcctgacaaaagttttgaaagtggtgtagttctgttcctggaacagttcatcagatactcggaggtaggtgaatccgtgcaacttgtgttctggagggccattcttgttgatgccttgaggtcttgcattcctgaggatggtgttgtaagctgttgcatcatatcggttgagtgcattttcacatgccaaatttaggccctctctccatccagcactcagcacctgttgaaccagttcctcaggtgcacttttcgcctctgaactctgttcactgtccctcatctcagcacaagtgaagttcatgctagcacgatgccttgtgagcatgtgtgctatggttctgtagcggatccagcctgcttttttgtacaaacttgt

SEQ ID NO: 12 - DNA, 1985 bp, Artificial Sequence
  maize PWD RNAi cassette
acaagtttgtacaaaaaagcaggctggatccgatctactgctaatgtggaggatttggctggaatgtctgctgctggactctatgaatcaattcctaatgtcagtctctctgaccctagatcctttggagctgcggttggacaggtttgggcatcattatacacaaggagagcaatccttagccgtcgagcagccggtgtacctcagagagacgcaaagatggcggttctcgtgcaggaaatgctgcagccagatctctcttttgtgcttcatacaattagcccagttgaccatgatcccaaattagtagaggctgaggttgcccctggtcttggagaaaccctcgcttctctcgagacccagctttcttgtacaaagtggttggcgccaaagggcgaattcactagtaagcttgggcccgcggccgcaggtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttgttttttgaactgagaaaaatctttgggtttttggttggattctttcatggagtcgcgaccttccgtattcttctctttgatctccccgcttgcggattcataatattcggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgaggtatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcagggcgccaaccactttgtacaagaaagctgggtctcgagagaagcgagggtttctccaagaccaggggcaacctcagcctctactaatttgggatcatggtcaactgggctaattgtatgaagcacaaaagagagatctggctgcagcatttcctgcacgagaaccgccatctttgcgtctctctgaggtacaccggctgctcgacggctaaggattgctctccttgtgtataatgatgcccaaacctgtccaaccgcagctccaaaggatctagggtcagagagactgacattaggaattgattcatagagtccagcagcagacattccagccaaatcctccacattagcagtagatcggatccagcctgcttttttgtacaaacttgt

SEQ ID NO: 13 - DNA, 1985 bp, Artificial Sequence
  maize AMY3 RNAi cassette
acaagtttgtacaaaaaagcaggctggatccggaatgatcttaaagaatggctttgctggatgagaaaggaagtcggctacgatggatggagacttgactttgttcgtggtttctggggtggatatgtcaaggactatttggaagcaagtgaaccatactttgcagtaggagagtactgggactccctcagttatacttatggtgaaatggactacaatcaggatgcccacaggcagagaattgttgattggataaatgctacaaatggaactgctggcgcatttgatgttaccactaaaggaatacttcatgcggcgcttgaaagatcagagtattggcgcctgtccgatctcgagacccagctttcttgtacaaagtggttggcgccaaagggcgaattcactagtaagcttgggcccgcggccgcaggtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttgttttttgaactgagaaaaatctttgggtttttggttggattctttcatggagtcgcgaccttccgtattcttctctttgatctccccgcttgcggattcataatattcggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgaggtatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcagggcgccaaccactttgtacaagaaagctgggtctcgagatcggacaggcgccaatactctgatctttcaagcgccgcatgaagtattcctttagtggtaacatcaaatgcgccagcagttccatttgtagcatttatccaatcaacaattctctgcctgtgggcatcctgattgtagtccatttcaccataagtataactgagggagtcccagtactctcctactgcaaagtatggttcacttgcttccaaatagtccttgacatatccaccccagaaaccacgaacaaagtcaagtctccatccatcgtagccgacttcctttctcatccagcaaagccattctttaagatcattccggatccagcctgcttttttgtacaaacttgt

SEQ ID NO: 14 - DNA, 2009 bp, Zea mays
  5′UTR (1)..(2009): Zm015970 5′ UTR
ggcgctcccagccgtgaaaacgcgtatcgcgccaccaccggggacccctcccgcgccgggacgagagagctgatcgacggtcgctagaggagagaggaggtcgacagacgacaggggacggcgcgcgtcaggtggtggggctgaccgctgacggggacacgcgggggtgcgcgcgtcgtccgttcgatcgatctgtcgtcggcccggccgttcggcgagatagccaggccagagcttgccttgccgccggcggtgaaacggcgacagcttggctagagaacacgaggcaccgcgttttgatcgcgcctctcgtccccgggcaatcgctgttgacgagtatggccggcaggccgcggcacgcgagggccgtccagtttccggaggtaaggagggagcttcctactcttgtaggcatcgtccttttcctttgaaaatctgttgtatacgtttccttctttttcccttcaaacgactggtttatttacacatacatttcaggctccgcattttagacatcaaggaaaagtatcttttggagtatctccaacaatgccttaaactaaaatatgatactaaaaactactccaacagtgcatcattttataaaatttggttaaaattttatagggcgttctctcaagtaactcaaatatactacaccgcagtgatctgtcctataatctagatgtgaggttttaatattgaaactgaatgttttgttggtgacctaaattctataaaatatactcattttaaattatagagcacttttataggtcattttgttagagatgatcttatacattgtgtttaaccgcagcaatcggctgtggatttatttgtttttagatttgggcaatatgtttgtatagacttcgttttattgttgaattattcaaagctagctagagcaactgatcatatactcgtatgagtctaacacatgttcatgccctacaaaggaagaaagtctaacacatgttcatgttcagaattgagagaaaaaaagaaacttttagctacagtacaacttacaagtcttgcaggaacacaggatgcagcctacattacatatacgtctctcttggtcacaagacggcatcctgtgatgagaatatttccaggaagattcttttgtcaatttctagccttgcagaaattctcggtaagctattttcagacttgtcacaatttaagtcgacatcacaaacaaagcaaaaaaaaacgcagtccctcattgttgtgatttgtgaataagaaaatcgtgtggagaaatttcaagggaatgtgttgtggcacaaataaactttgtagatgtacaaaaatgcaaaggggattagtacaggtaggactactcaattaaataacatttgaaattaaattttttcactaaaataaatattcattgttccacggtcttattgtggacactaaagtgccgtttgattcacaaaatgtaacgtaaatgataacggtaatgctttacactcgatatcagcggtaacaagtttgaataagacaatatcagttttgtaaacggttggatttaacaaacatggttgtgtgcatgcatgcgagagggggagacgaggggcgaaacgaggatccaaaattagggggttaaattagattaaggggctgttagaagtgttagaagcatttttacattatttacatgagaattagcttaaaaactaacatgtcctatagaattaagcattaaaaccgcatgagtaataaatatataatacaaatacaaaaaaaaaactgagaagaaatggcacagatccgaagtggcaaattgtcttccttcgccttcgcgcgctcttctataaatacctcccctctcaccattcccttgccatcagacaagagacacacacacaccacacgcagtcacgcacagcctcggcctccgcagctgcaggcgcctgctgctcttcgacaagaccccaagaactcgccaacctaacctaggtcagccacccttccttctaccttctgcgaca

SEQ ID NO: 15 - DNA, 194 bp, Figwort mosaic virus
  enhancer (1)..(194): FMV enhancer
agctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaa

SEQ ID NO: 16 - DNA, 293 bp, Cauliflower mosaic virus
  enhancer (1)..(293): CaMV 35S enhancer
acttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttc

SEQ ID NO: 17 - DNA, 1166 bp, Oryza sativa
  Intron (1)..(1166): rice sucrose synthase intron loop
gtatgttgcttccattgccaaactgttcccttttacccataggctgattgatcttggctgtgtgattttttgcttgggtttttgagctgattcagcggcgcttgcagcctcttgatcgtggtcttggctcgcccatttcttgcgattctttggtgggtcgtcagctgaatcttgcaggagtttttgctgacatgttcttgggtttactgctttcggtaaatctgaaccaagaggggggtttctgctgcagtttagtgggtttactatgagcggattcggggtttcgaggaaaaccggcaaaaaacctcaaatcctcgacctttagttttgctgccacgttgctccgccccattgcagagttctttttgcccccaaatttttttttacttggtgcagtaagaatcgcgcctcagtgattttctcgactcgtagtccgttgatactgtgtcttgcttatcacttgttctgcttaatcttttttgcttcctgaggaatgtcttggtgcctgtcggtggatggcgaaccaaaaatgaagggtttttgttttttgaactgagaaaaatctttgggtttttggttggattctttcatggagtcgcgaccttccgtattcttctctttgatctccccgcttgcggattcataatattcggaacttcatgttggctctgcttaatctgtagccaaatcttcatatctccagggatctttcgctctgtcctatcggatttaggaattaggatctaactggtgctaatactaaagggtaatttggaaccatgccattataattttgcaaagtttgaggtatgccatcggtatctcaatgatacttactaaaacccaacaaatccatttgataaagctggttcttttatccctttgaaaacattgtcagagtatattggttcaggttgatttattttgaatcagtactcgcactctgcttcgtaaaccatagatgctttcagttgtgtagatgaaacagctgtttttagttatgttttgatcttccaatgcttttgtgtgatgttattagtgttgatttagcatggctttcctgttcagagatagtcttgcaatgcttagtgatggctgttgactaattattcttgtgcaagtgagtggttttggtacgtgttgctaagtgtaacctttctttgcag

SEQ ID NO: 18 - DNA, 25 bp, Artificial Sequence
  AttB1 site
acaagtttgtacaaaaaagcaggct

SEQ ID NO: 19 - DNA, 25 bp, Artificial Sequence
  AttB2 site
acccagctttcttgtacaaagtggt

SEQ ID NO: 20 - DNA, 1009 bp, Zea mays
  terminator (1)..(1009): Zm015970 terminator
cgccttgcattaattggcgtctagatggcctgccctcatcagttcaccgcggatcagctcggtgctcgaagatgacagcttatgacctttgagaatgtctactaagaatcgccctttgtataatactactagatcctctcttttttgtattttctttcctaggactagcttccagtgttactgggctcatttcgtggtgtttctattatgactaagattagagagagaatgagagatggatccatctaaataaaggttccagattttttttcccctctctgtccttctgttgcctccgaattctgcggtccaaaacaacgcgtggctgagaatgcatcgggatgatagtatgatacccattccattcgtcatgtttcacgaggccaggaacggcagccgtgtaaaggtctggtcgcccaccttgtcctcttgctggtgactgacaggcacaggggagcatggctgtccctgacgggaagcgccgtgcaatttgcacactgagatcttcgcgatggattcagcgttggagcgtagctgatctttctttgtgcgcgcttgctgactgctcctttggctcggcacacttttgttgttcagcatgggcttttggcagagctgatatgccatcctcgcaggcgggtgcaacctttggtgctctatgggccgttgcattttagattccggttcagatgtatctttcgtgcttttttcagaagttcgtcgaagataccaaatcctttggtgttagccgctcgtgattgtgggtaaaaggcctaaatcttcaaactaaacgacagtcctttattactaagctgcattttccatctttagtgcttactacacatgcacggtacattcttatactcctttgagcacacgcatttaaaatgtgccacgtgagcaccgtgccttttaaacacaagatgcattgccttcgaaacagctctagccgacttgaaaggtcaagaacacagttcagttttccttatatatataataaaatgtaaaa